(19) United States
(12) Patent Application Publication
(10) Pub. No.: US 2006/0034846 A1
(43) Pub. Date: Feb. 16, 2006

(54) Use of TF antagonists

(76) Inventors: Mirella Ezban, Copenhagen K (DK); Nathalie Busso, Belmont (CH)

Correspondence Address: NOVO NORDISK, INC.;PATENT DEPARTMENT, 100 COLLEGE ROAD WEST, PRINCETON, NJ, 08540, US

(21) Appl. No.: 10/822,943

(22) Filed: Apr. 12, 2004

Related U.S. Application Data
(60) Provisional application No. 60/462,764, filed on Apr. 14, 2003.

(51) Int. Cl. A61K 39/395; A61K 38/37

(52) U.S. Cl. 424/145.1; 514/12

(57) ABSTRACT

Methods of inducing, promoting, and/or enhancing one or more physiological responses associated with the treatment and/or prevention of inflammatory arthritides and/or other inflammation-related conditions in mammals and/or mammalian tissues including delivery of an effective amount of at least one tissue factor antagonist, at least one tissue factor inhibitor, or a combination thereof to a mammal and/or mammalian tissue under conditions such that such a physiological response is induced, promoted, and/or enhanced.

### FIELD OF THE INVENTION

This invention relates to novel use of tissue factor antagonists in the prophylaxis and/or treatment of diseases or disorders.

### BACKGROUND OF THE INVENTION

Tissue Factor (TF) is a cellular transmembrane receptor for plasma coagulation factor VIIa (FVIIa) and formation of TF/FVIIa complexes on the cell surface triggers the coagulation cascade in vivo. The TF/FVIIa complex efficiently activates coagulation factors IX and X. The resultant protease factor Xa (FXa), activates prothrombin to thrombin, which in turn converts fibrinogen into a fibrin matrix. Normally, TF is constitutively expressed on the surface of many extravascular cell types that are not in contact with the blood, such as fibroblasts, pericytes, smooth muscle cells and epithelial cells, but not on the surface of cells that come in contact with blood, such as endothelial cells and monocytes.

FVIIa is a two-chain, 50 kilodalton (kDa) vitamin-K dependent, plasma serine protease which participates in the complex regulation of in vivo hemostasis. FVIIa is generated from proteolysis of a single peptide bond from its single chain zymogen, Factor VII (FVII), which is present at approximately 0.5 μg/ml in plasma. The zymogen is catalytically inactive. The conversion of zymogen FVII into the activated two-chain molecule occurs by cleavage of an internal peptide bond. In the presence of calcium ions, FVIIa binds with high affinity to exposed TF, which acts as a cofactor for FVIIa, enhancing the proteolytic activation of its substrates FVII, Factor IX and FX.

Inhibition of the catalytic activity of the TF/FVIIa complex in vivo occurs through formation of a quaternary complex between TF, FVIIa, TF pathway inhibitor (TFPI), and FXa. Under normal conditions, TFPI is synthesized primarily by endothelial cells, although activated monocytes and stimulated fibroblasts can also synthesize TFPI. In vivo, the major pool (80%) of TFPI is bound to glycosaminoglycan binding sites on the endothelial cell surface, and the remaining TFPI is associated with plasma lipoproteins or is stored in platelets.

Inactivated FVII (FVIIai) is FVIIa modified in such a way that it is catalytically inactive. Thus, FVIIai is not able to catalyze the conversion of FX to FXa, but is still able to bind tightly to TF in competition with active endogenous FVIIa and thereby inhibit the TF function.

### BRIEF SUMMARY OF THE INVENTION

The invention provides new and useful methods of inducing, promoting, and/or enhancing one or more physiological responses associated with the amelioration, reduction, cessation, and/or prevention of inflammatory arthritides or related conditions in mammals and/or mammalian tissues comprising delivering (e.g., by direct administration or expression from an administered gene transfer vector) an effective amount of a TF antagonist and/or TF inhibitor (e.g., an siRNA against TF; an antisense nucleic acid which reduces TF production; or a nucleic acid that otherwise downregulates TF gene expression, translation, or TF production (e.g., by knocking out endogenous TF promoter(s) and/or by delivering a peptide that downregulates endogenous TF expression)) to a mammalian tissue (in vitro, in vivo, and/or ex vivo) comprising TF presenting cells and being associated with an inflammatory arthritic condition or risk thereof (in the same tissue or other but pathologically related tissue) under conditions such that a physiological condition associated with the amelioration, reduction, cessation (cure), and/or prevention of at least one inflammatory arthritic condition is induced, enhanced, and/or promoted therein. The inventive methods are useful in, among other things, the treatment of inflammatory arthritic conditions in human patients diagnosed as having or of being at substantial risk of soon developing such conditions. These and other advantages of the invention, as well as additional inventive features, will be apparent from the description of the invention provided herein.

### DESCRIPTION OF THE INVENTION

The invention described herein provides a method of inducing, promoting, and/or enhancing one or more physiological responses associated with inflammatory arthritides therapy (e.g., as part of a regimen for an inflammatory arthritis) or prevention in a mammal (e.g., a human patient identified as being in need of such treatment) comprising administering an effective amount of a TF antagonist and/or TF inhibitor to the mammal under conditions such that a physiological condition associated with a therapeutic regimen for and/or prevention of at least one arthritic condition is (preferably detectably) induced, enhanced, and/or promoted. In one exemplary aspect, the invention also or alternatively provides a method of reducing inflammation in a mammal afflicted with one or more arthritic conditions by the practice of such a method. In another exemplary aspect, the invention provides a method of reducing the severity, spread, onset time, and/or risk of developing or occurrence of an arthritic condition by such a method. In yet another exemplary aspect, the invention provides a method of treating inflammatory arthritides (as further defined herein) comprising performing such a method.

The inventive methods can be performed with any suitable type of TF antagonist and/or TF inhibitor. Although each distinct aspects of the invention, it should be understood that methods described herein with reference to TF antagonists can be practiced with a suitable TF inhibitor, and vice versa, unless otherwise stated or clearly contradicted by context. In general, the use of a TF antagonist (alone or in combination with a suitable TF inhibitor and/or other agents) is preferred in the practice of the various inventive methods described herein. The term “TF antagonist” refers to any compound capable of binding directly to TF with a sufficiently high affinity, specificity, and activity so as to inhibit the conversion of FX to FXa in an FXa generation assay. Examples of TF antagonists include, but are not limited to, FVIIai and inhibitory antibodies against TF. The TF antagonist also desirably is capable of binding a number of TF presenting cells in vitro and/or in vivo. TF presenting cells include cells wherein TF is expressed and presented on the cell membrane and cells that possess a cell membrane associated with TF peptides that were expressed by another cell.

Desirably, the TF antagonist employed in the inventive methods is not cytotoxic. The term “cytotoxic” refers substances that inhibit or prevent major function(s) of cells and/or causes significant damage and/or destruction to cells. Desirably, the TF antagonist also or alternatively is capable of binding TF with high affinity and specificity but does not initiate blood coagulation. The TF antagonist can comprise one or more than one binding site for TF. In some aspects, it is desirable for the TF antagonist to comprise more than one binding site for TF.

In an exemplary aspect, the TF antagonist is factor FVIIa polypeptide chemically inactivated in the active site. Desirably, such an FVIIa has an affinity for TF that is at least about as great as native FVI a. Alternatively, the TF antagonist can be a catalytically impaired FVIIa mutant. Desirably, such modified FVIIa peptides have an in vivo half life of at least about 2-3 hours. Derivatives of such peptides may have longer half-lives through conjugation of particular chemical moieties, by formulation in a pharmaceutically acceptable composition with one or more stabilizing chemicals, and/or by combining additional stabilizing amino acid sequences (e.g., as a fusion protein).

The term “active site” when used herein with reference to FVIIa refers to the catalytic and zymogen substrate binding site, including the “S₁” site of FVIIa as that term is defined by Schecter, I. and Berger, A., (1967) Biochem. Biophys. Res. Commun. 7:157-162.

In one aspect of the invention, the TF antagonist is an inactive FVIIa polypeptide. In a more particular exemplary aspect, the TF antagonist is one or more chemically inactivated FVII molecules in which the active site is covalently modified by application of one or more covalent active site inhibitors. The inactivation of FVIIa proteolytic activity may be obtained in vitro by application of a suitable covalent active site inhibitor, e.g., a chloromethyl ketone. Such TF antagonists can have very high affinity for TF as compared to the binding of native FVII. Such high affinity can provide a more efficacious and safe treatment of a patient in need thereof. The TF antagonist may also have a higher affinity for TF due an avidity effect in dimers, trimers, or other multimers with multiple TF binding sites.

A FVII peptide that is “catalytically inactivated in the active site” refers to an FVIIa peptide wherein an FVIIa inhibitor is bound to the FVIIa polypeptide and decreases or prevents the FVIIa-catalyzed conversion of FX to FXa. An FVIIa inhibitor may be identified as a substance, which reduces the amidolytic activity by at least 50% at a concentration of the substance at 400 μM in the FVIIa amidolytic assay described by Persson et al. (Persson et al., J. Biol. Chem. 272: 19919-19924 (1997)). Preferred are substances reducing the amidolytic activity by at least 50% at a concentration of the substance at 300 μM; more preferred are substances reducing the amidolytic activity by at least 50% at a concentration of the substance at 200 μM.

Such an “FVIIa inhibitor” may be selected from any one of several groups of FVIIa directed inhibitors. Such inhibitors are broadly categorized for the purpose of the present invention into i) inhibitors which reversibly bind to FVIIa and are cleavable by FVIIa, ii) inhibitors which reversibly bind to FVIIa but cannot be cleaved, and iii) inhibitors which irreversibly bind to FVIIa. For a review of inhibitors of serine proteases see Proteinase Inhibitors (Research Monographs in cell and Tissue Physiology; v. 12) Elsevier Science Publishing Co., Inc., New York (1990).

An FVIIa inhibitor moiety may also be an irreversible FVIIa serine protease inhibitor. Such irreversible active site inhibitors generally form covalent bonds with the protease active site. Such irreversible inhibitors include, but are not limited to, general serine protease inhibitors such as peptide chloromethylketones (see, Williams et al., J. Biol. Chem. 264:7536-7540 (1989)) or peptidyl cloromethanes; azapeptides; acylating agents such as various guanidinobenzoate derivatives and the 3-alkoxy-4-chloroisocoumarins; sulphonyl fluorides such as phenylmethylsulphonylfluoride (PMSF); diisopropylfluorophosphate (DFP); tosylpropylchloromethyl ketone (TPCK); tosyllysylchloromethyl ketone (TLCK); nitrophenylsulphonates and related compounds; heterocyclic protease inhibitors such as isocoumarines, and coumarins.

Examples of peptidic irreversible FVIIa inhibitors include, but are not limited to,
Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone.

Examples of FVIIa inhibitors also include benzoxazinones or heterocyclic analogues thereof such as described in PCT/DK99/00138.

Examples of other FVIIa inhibitors include, but are not limited to, small peptides such as for example Phe-Phe-Arg, D-Phe-Phe-Arg, Phe-Phe-Arg, D-Phe-Phe-Arg, Phe-Pro-Arg, D-Phe-Pro-Arg, Phe-Pro-Arg, D-Phe-Pro-Arg, L- and D-Glu-Gly-Arg; peptidomimetics; benzamidine systems; heterocyclic structures substituted with one or more amidino groups; aromatic or heteroaromatic systems substituted with one or more C(═NH)NHR groups in which R is H, C₁₋₃alkyl, OH or a group which is easily split of in vivo.

In a particular exemplary aspect, an FVIIa polypeptide used in one of the inventive methods described herein is catalytically inactivated in the active site with a chloromethyl ketone inhibitor independently selected from Phe-Phe-Arg chloromethyl ketone, Phe-Phe-Arg chloromethylketone, D-Phe-Phe-Arg chloromethyl ketone, D-Phe-Phe-Arg chloromethylketone Phe-Pro-Arg chloromethylketone, D-Phe-Pro-Arg chloromethylketone, Phe-Pro-Arg chloromethylketone, D-Phe-Pro-Arg chloromethylketone, L-Glu-Gly-Arg chloromethylketone and D-Glu-Gly-Arg chloromethylketone, Dansyl-Phe-Phe-Arg chloromethyl ketone, Dansyl-Phe-Phe-Arg chloromethylketone, Dansyl-D-Phe-Phe-Arg chloromethyl ketone, Dansyl-D-Phe-Phe-Arg chloromethylketone, Dansyl-Phe-Pro-Arg chloromethylketone, Dansyl-D-Phe-Pro-Arg chloromethylketone, Dansyl-Phe-Pro-Arg chloromethylketone, Dansyl-D-Phe-Pro-Arg chloromethylketone, Dansyl-L-Glu-Gly-Arg chloromethylketone, Dansyl-D-Glu-Gly-Arg chloromethylketone, and combinations of any thereof.

In another aspect, the TF antagonist is full length FVII or another active FVII peptide. For example, in an exemplary aspect, the TF antagonist is FVII (des-Gla) or another TF-binding FVII derived protein (including truncated forms, analogs, derivatives and fusion proteins (monomers, homo- or heterodimers or multimers)). The different affinity of such molecules for TF may provide a method for reducing the potentially undesirable effect on general hemostasis of some FVII peptides.

The terms “FVIIa polypeptide” or “FVIIa polypeptides” as used herein refer to native Factor VIIa, as well as equivalents of Factor VIIa that contain one or more amino acid sequence alterations relative to native Factor VIIa (i.e., Factor VII variants), and/or contain truncated amino acid sequences relative to native Factor VIIa (i.e., Factor VIIa fragments), but which substantially retain FVIIa activity with respect to TF in a mammalian tissue. Such equivalents may exhibit different properties relative to native Factor VIIa, including stability, phospholipid binding, altered specific proteolytic activity, and the like. Unless otherwise indicated or contradicted by context, the terms “Factor VII” or “FVII” also can refer to Factor VII polypeptides in their uncleaved (zymogen) form. The terms “Factor VIIa” or “FVIIa” are intended to mean native bioactive forms of FVII. Typically, FVII is cleaved between residues 152 and 153 to yield FVIIa. The term “Factor VIIa” is also intended to encompass, without limitation, polypeptides having the amino acid sequence 1-406 of wild-type human Factor VIIa (as disclosed in U.S. Pat. No. 4,784,950), as well as wild-type Factor VIIa derived from other species, such as, e.g., bovine, porcine, canine, murine, and salmon Factor VIIa. It further encompasses natural allelic variations of Factor VIIa that may exist and occur from one individual to another. Also, degree and location of glycosylation or other post-translation modifications in FVII peptides may vary depending on the chosen host cells and the nature of the host cellular environment. Peptides having such cell-specific translational variations are intended to be encompassed by these definitions.

The terms “variant” or “variants”, as used herein, are intended to designate peptides, polypeptides, and/or proteins (which terms are used interchangeably throughout unless otherwise indicated) (e.g., human Factor VII) comprising a sequence that is substantially similar to a native peptide (e.g., native human coagulation Factor VII) in terms of amino acid sequence identity (i.e., a variant is a peptide wherein one or more amino acids of the native “parent peptide” or “parent protein” have been substituted by another amino acid; one or more amino acids of the parent protein have been deleted; one or more amino acids have been inserted in protein; and/or wherein one or more amino acids have been added to the parent protein). Unless otherwise indicated, additions can take place either at the N-terminal end or at the C-terminal end of the parent protein or both. Typically, a variant (e.g., a FVII variant) has a total amount of amino acid substitutions and/or additions and/or deletions independently selected from the group consisting of 1, 2, 3, 4, 5, 6, 7, 8, 9, and 10 amino acids.

A number of potentially suitable FVII peptides are known in the art. International patent applications WO 92/15686, WO 94/27631, WO 96/12800, WO 97/47651 describe FVIIai. International patent applications WO 90/03390, WO 95/00541, WO 96/18653, and European Patent EP 500800 describe peptides derived from FVIIa having TF/FVIIa antagonist activity. International patent application WO 01/21661 relates to a bivalent inhibitor of FVII and FXa. Hu Z and Garen A (2001) Proc. Natl. Acad. Sci. USA 98; 12180-12185, Hu Z and Garen A (2000) Proc. Natl. Acad. Sci. USA 97; 9221-9225, Hu Z and Garen A (1999) Proc. Natl. Acad. Sci. USA 96; 8161-8166, and International patent application WO 0102439 relates to immunoconjugates which comprises the Fc region of a human IgG1 immunoglobulin and a mutant FVII polypeptide, that binds to TF but do not initiate blood clotting.

In general, it should be noted that peptides described herein can, unless otherwise indicated, comprise or refer to “natural”, i.e., naturally occurring amino acids as well as “non classical” D-amino acids including, but not limited to, the D-isomers of the common amino acids, α-isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Ca-methyl amino acids, Nα-methyl amino acids, and amino acid analogues in general. In addition, the amino acids can include Abu, 2-amino butyric acid; γ-Abu, 4-aminobutyric acid; ε-Ahx, 6-aminohexanoic acid; Aib, 2-amino-isobutyric acid; β-Ala, 3-aminopropionic acid; Orn, ornithine; Hyp, trans-hydroxyproline; Nle, norleucine; Nva, norvaline. The three-letter indication “GLA” as used herein means 4-carboxyglutamic acid (γ-carboxyglutamate).

As used herein, “Factor VII equivalent” encompasses, without limitation, any suitable equivalent of Factor VIIa exhibiting TF binding activity. The term “TF binding activity” as used herein means the ability of an FVIIa polypeptide or TF antagonist to inhibit the binding of recombinant human ¹²⁵I-FVIIa to cell surface human TF. The TF binding activity may be measured as described in Assay 3 herein.

Factor VII equivalents also include proteolytically inactive variants of FVIIa. In an exemplary aspect, the TF antagonist is a human FVIIa-derived peptide, which comprises an FVII amino acid sequence comprising an amino acid substitution of the lysine corresponding to position 341 of native human coagulation Factor VII. In another aspect, the TF antagonist is a human FVIIa-derived peptide, which comprises an FVII amino acid sequence that has an amino acid substitution of the serine corresponding to position 344 of native human coagulation Factor VII. In another aspect, the TF antagonist is a human FVIIa-derived peptide, which comprises an FVII sequence that also or alternatively has an amino acid substitution of the aspartic acid corresponding to position 242 of native human coagulation Factor VII. In yet another aspect, the TF antagonist is a human FVIIa-derived peptide, which comprises an FVII amino acid sequence that also or alternatively has an amino acid substitution of the histidine corresponding to position 193 of native human coagulation Factor VII. Such peptides can correspond to native FVII in length, correspond to active FVII fragments, or be fusion proteins comprising a full length or truncated FVII sequence modified as indicated. In an exemplary aspect, the TF antagonist is FVII-(K341A), FVII-(S344A), FVII-(D242A), and/or FVII-(H193A).

Terminology used to describe specific amino acid substitutions in certain aspects of the invention is as follows. The first letter represents the amino acid naturally present at a position of native human coagulation Factor VII. The following number represents the position in native human coagulation Factor VII. The second letter represents the different amino acid substituting for the natural amino acid. An example is FVII-(K341A), where a lysine at position 341 of native human coagulation Factor VII is replaced by an alanine. In another example, FVII-(K341A/S344A), the lysine at position 341 of native human coagulation Factor VII is replaced by an alanine and the serine in position 344 of native human coagulation Factor VII is replaced by an alanine in the same Factor VII polypeptide.

In another aspect, the invention provides a method of modulating a TF/FVIIa mediated or associated process in TF presenting cells contained in a tissue associated with an inflammatory condition comprising administering an effective amount of a TF inhibitor and/or TF antagonist such that the TF/FVIIa mediated or associated process is modulated and the associated inflammation is detectably reduced. A TF/FVIIa mediated or associated process or event, or a process or event associated with TF-mediated coagulation activity, is any event, which requires the presence of TF/FVIIa.

The term “TF-mediated coagulation activity” means coagulation initiated by TF through the formation of the TF/FVIIa complex and its activation of FIX and Factor X to FIXa and FXa, respectively. TF-mediated coagulation activity is measured in an FXa generation assay. The term “FXa generation assay” as used herein is intended to mean any assay where activation of FX is measured in a sample comprising TF, FVIIa, FX, calcium and phospholipids. An example of an FXa generation assay is described in Assay 1 herein.

Such processes or events include, but are not limited to, formation of fibrin which leads to thrombus formation; platelet deposition; proliferation of smooth muscle cells (SMCs) in the vessel wall, such as, for example, in intimal hyperplasia or restenosis, which is thought to result from a complex interaction of biological processes including platelet deposition and thrombus formation, release of chemotactic and mitogenic factors, and the migration and proliferation of vascular smooth muscle cells into the intima of an arterial segment; and deleterious events associated with post-ischemic reperfusion, such as, for example, in patients with acute myocardial infarction undergoing coronary thrombolysis.

The no-reflow phenomenon, that is, lack of uniform perfusion to the microvasculature of a previously ischemic tissue has been described for the first time by Krug et al., (Circ. Res. 1966; 19:57-62).

The general mechanism of blood clot formation is reviewed by Ganong, in Review of Medical Physiology, 13^{th} ed., Lange, Los Altos Calif., pp 411-414 (1987). Coagulation requires the confluence of two processes, the production of thrombin which induces platelet aggregation and the formation of fibrin which renders the platelet plug stable. A process mediated by or associated with TF/FVIIa, or an TF-mediated coagulation activity, can include any step in the coagulation cascade from the formation of the TF/FVIIa complex to the formation of a fibrin platelet clot and which initially requires the presence of TF/FVIIa. For example, the TF/FVIIa complex initiates the extrinsic pathway by activation of FX to FXa, FIX to FIXa, and additional FVII to FVIIa. TF/FVIIa mediated or associated process, or TF-mediated coagulation activity can be conveniently measured employing standard assays such as those described in Roy, S., (1991) J. Biol. Chem. 266:4665-4668, and O'Brien, D. et al., (1988) J. Clin. Invest. 82:206-212 for the conversion of FX to FXa in the presence of TF/FVIIa and other necessary reagents.

In another aspect, the TF antagonist used in the various methods of the invention is an antibody against TF. Typically, the TF antibody will comprise one or more monoclonal antibodies against TF; an active fragment of such an antibody; or a derivative thereof (polyclonal antibodies against TF may also be useful). In another exemplary aspect, the antibody is a human monoclonal antibody. In an additional aspect, the antibody is an antibody against human TF. Methods of preparing human antibodies against human TF are described in, e.g., U.S. Patent Application 2004-0001830, International Patent Application PCT/DK02/00644, and Danish patent application PA 2001 01437.

The invention provides methods for inducing, promoting, and/or enhancing one or more physiological effects associated with the amelioration and/or cure of inflammatory arthritic conditions in a mammalian tissue comprising administering an effective amount of one or more TF antagonists to a tissue (e.g., a population of cells; an organ; etc.) comprising TF presenting cells associated with inflamed tissue (e.g., by proximity or by being the same tissue) under conditions such that at least one physiological response associated with the amelioration and/or cure of an inflammatory arthritic condition in such cells is detectably induced, promoted, and/or enhanced therein. In one aspect of the inventive method, the invention provides a therapeutic regimen against one or more inflammatory arthritic conditions in mammals. The inventive methods also or alternatively can be used to reduce the risk of likelihood of developing an arthritic condition (as compared to similar mammals and/or mammalian tissues—which may be, for example, determined through clinical trials or other clinical data), delaying the onset of an arthritic condition, reducing the spread of an arthritic condition, reducing the severity of an imminent arthritic condition, or otherwise preventing the occurrence of an arthritic condition (preferably in a human patient that has been identified as being at significant risk for near term development of such a condition).

In another aspect, the invention provides a method of treating inflammatory arthritides in mammals by, among possibly other things, delivering an effective amount of a TF antagonist to a mammal afflicted with or at substantial risk of developing an inflammatory arthritic condition. “Treatment”, in this respect, means the administration of an effective amount of a therapeutically active composition (e.g., a TF antagonist, a TF antagonist-expressing vector, and/or a TF antagonist in association with a pharmaceutically acceptable carrier) with the purpose of preventing any symptoms or disease state to develop or with the purpose of curing or easing such symptoms or disease states already developed. The term “treatment” is thus meant to include prophylactic treatment.

In an exemplary aspect, the invention provides a method for preventing or treating a disease or disorder associated with inflammation in a mammalian tissue (e.g., in one or more organs and/or organ-associated tissues in a human patient), which method comprises administering a therapeutically effective amount of a TF antagonist in combination with a pharmaceutical acceptable carrier, to a mammal in need of such a treatment.

In another illustrative aspect, the invention provides a method for preventing or treating a disease or disorder associated with inflammatory arthritis comprising contacting a TF presenting cell in a tissue comprising such a cell and being associated with an arthritic condition in a mammal with an effective amount of a TF antagonist (either directly or by any other suitable technique—such as expression of a TF antagonist from a suitable gene transfer vector).

An “effective amount” is any amount of the TF antagonist, TF inhibitor, combination thereof, or related composition that is sufficient to detectably induce, promote, enhance or otherwise bring about one or more desired physiological effects (e.g., the reduction of inflammation; the reduction of risk of developing an inflammatory condition as compared with a population of similar mammals not receiving the indicated regimen; etc.).

In another aspect, the various inventive methods described herein also or alternatively practiced by, among other things, delivering an effective amount of a TNF inhibitor to a tissue comprising TF presenting cells and being associated with a tissue afflicted with an inflammatory arthritic condition or at risk of developing such a condition. Any suitable TF inhibitor can be used. A TF inhibitor can be, for example, a nucleic acid that regulates TF expression (e.g., an anti-TF siRNA; an anti-TF antisense molecule; etc.); a protein that negatively regulates TF expression, translation, and/or production in mammals (e.g., an interleukin-10) or a gene transfer vector capable of producing such a peptide in mammalian cells; or a small molecule that negatively impacts TF expression, translation, and/or production in mammalian cells (e.g., nicotinamide, fenofibric acid, adenosine, PPARa agonists, and the like). The inventive methods also or alternative can include a step of downregulating production of other endogenous factors that increase TF in mammalian cells.

The inventive method of the invention can be useful in the amelioration, cessation, remediation, and/or prevention of a number of diseases and disorders. Thus, in one aspect, the inventive method is applied to treat a rheumatic disorder (e.g., rheumatoid arthritis, osteoarthritis, juvenile (rheumatoid) arthritis, seronegative polyarthritis, ankylosing spondylitis, reiter's syndrome and reactive arthritis, psoriatic arthritis, enteropathic arthritis, polymyositis, dermatomyositis, scleroderma, systemic sclerosis, vasculitis, cerebral vasculitis, Lyme disease, staphylococcal-induced (“septic”) arthritis, sjogren's syndrome, rheumatic fever, polychondritis and polymyalgia rheumatica, giant cell arteritis, fibromyelgia, or any combination thereof). The inventive methods of the invention may be particularly useful in the treatment of rheumatoid arthritis (RA) and osteoarthritis. In another aspect, the method provides a method of ameliorating, ceasing, reducing, or preventing a condition associated with or characterized by chronic synovitis in a mammalian host.

As described elsewhere herein, the inventive methods can be used to ameliorate, substantially eliminate, or even eliminate within means of detection and/or sensation an inflammatory condition. Accordingly, the inventive methods can also be used as a treatment for inflammatory related conditions such as in combating graft versus host rejection; septic shock; ameliorating and/or reducing side effects from radiation therapy; treating temporal mandibular joint disease; or otherwise treating an inflammatory condition resulting from strain, sprain, cartilage damage, trauma, orthopedic surgery, infection, or other disease processes, or combination of any thereof.

The inventive methods also can also or alternatively can be used in the treatment of cachexia/anorexia; chronic fatigue syndrome, or depression.

The inventive methods also or alternatively can be used in the treatment of diabetes (e.g., juvenile onset Type 1 and diabetes mellitus).

In another aspect, the invention provides a method of treating inflammatory bowel disease comprising delivering an effective amount of a TF antagonist and/or TF inhibitor to a mammal diagnosed as having or being at substantial risk of developing inflammatory bowel disease (e.g., the invention provides a therapeutic regimen against IBD in a human patient diagnosed as suffering therefrom comprising delivering an effective amount of a TF antibody thereto).

The inventive methods also can be used to ameliorate, reduce, cure, or prevent ischemic injury, including cerebral ischemia (e.g., brain injury as a result of trauma, epilepsy, hemorrhage or stroke, each of which may lead to neurodegeneration);

In additional aspects, the inventive methods can be used to treat lung diseases (e.g., ARDS and pulmonary fibrosis); multiple sclerosis, ocular diseases; pancreatitis; reperfusion injury; and/or combinations of any thereof.

In further aspects, the method provides a method of treating pain in a mammalian host comprising delivering an effective amount of a TF inhibitor and/or TF antagonist to a tissue comprising TF presenting cells in the mammal under conditions such that the pain of the mammal is detectably reduced. In a particular aspect, the invention provides a method for improving the quality of life of a human patient suffering from inflammation-related pain comprising delivering an effective amount of a TF antagonist and/or TF inhibitor to such a tissue. Thus, the inventive method provides a method of inducing, promoting, and/or enhancing an analgesic effect in a mammalian host. In another aspect, the inventive method provides a method for reducing, curing, and/or preventing hyperalgesia in a mammalian host.

The invention also relates to a method of preparing TF antagonists as mentioned above. The TF antagonist may be produced by recombinant DNA techniques. To this end, nucleic acid sequences encoding human FVIIa may be isolated by preparing, for example, a genomic or cDNA library and screening for DNA sequences coding for all or part of the protein by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the nucleic acid sequence encoding the protein is preferably a DNA sequence of human origin, i.e., derived from a human genomic DNA or cDNA library.

DNA sequences encoding the human FVIIa polypeptides may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors. DNA sequences may also be prepared by polymerase chain reaction using specific primers, for instance as described in U.S. Pat. No. 4,683,202, Saiki et al., Science 239 (1988), 487-491, or Sambrook et al., supra.

DNA sequences encoding the human FVIIa polypeptides are usually inserted into a suitable vector, which may conveniently be subjected to recombinant DNA procedures. Thus, the vector may be an autonomously replicating vector that exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the human FVIIa polypeptides is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g., transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the human FVIIa polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. VIak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpia promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the human FVIIa polypeptides may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may also contain a set of RNA splice sites located downstream from the promoter and upstream from the insertion site for the FVIIa sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 EIb region, the human growth hormone gene terminator (DeNoto et al. Nuc. Acids Res. 9:3719-3730, 1981) or the polyadenylation signal from the human FVII gene or the bovine FVII gene. The expression vectors may also include a noncoding viral leader sequence, such as the adenovirus 2 tripartite leader, located between the promoter and the RNA splice sites; and enhancer sequences, such as the SV40 enhancer.

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct the human FVIIa polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the human FVIIa polypeptides in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed human FVIIa polypeptides into the secretory pathway of the cell. The signal peptide may be naturally-occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the human FVIIa polypeptides. The function of the leader peptide is to allow the expressed peptide to be directed from the endoplasmic reticulum to the Golgi apparatus and further to a secretory vesicle for secretion into the culture medium (i.e. exportation of the human FVIIa polypeptides across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the human FVIIa polypeptides, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the human FVIIa polypeptides of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the human FVIIa polypeptide of interest.

The host cell into which the DNA sequences encoding the human FVIIa polypeptides is introduced may be any cell, which is capable of producing the posttranslational modified human FVIIa polypeptides and includes yeast, fungi and higher eucaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk⁻ ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk⁻ ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the human FVIIa polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the human FVIIa polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The human FVIIa polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

For the preparation of recombinant human FVIIa polypeptides, a cloned wild-type FVIIa DNA sequence is used. This sequence may be modified to encode a desired FVIIa variant. The complete nucleotide and amino acid sequences for human FVIIa are known. See U.S. Pat. No. 4,784,950, which is incorporated herein by reference, where the cloning and expression of recombinant human FVIIa is described. The bovine FVIIa sequence is described in Takeya et al., J. Biol. Chem, 263:14868-14872 (1988), which is incorporated by reference herein.

The amino acid sequence alterations may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984). Thus, using the nucleotide and amino acid sequences of FVII, one may introduce the alterations of choice.

DNA sequences for use within the present invention will typically encode a pre-pro peptide at the amino-terminus of the FVIIa protein to obtain proper post-translational processing (e.g. gamma-carboxylation of glutamic acid residues) and secretion from the host cell. The pre-pro peptide may be that of FVIIa or another vitamin K-dependent plasma protein, such as factor IX, factor X, prothrombin, protein C or protein S. As will be appreciated by those skilled in the art, additional modifications can be made in the amino acid sequence of FVIIa where those modifications do not significantly impair the ability of the protein to act as a coagulation factor. For example, FVIIa in the catalytic triad can also be modified in the activation cleavage site to inhibit the conversion of zymogen FVII into its activated two-chain form, as generally described in U.S. Pat. No. 5,288,629, incorporated herein by reference.

Within the present invention, transgenic animal technology may be employed to produce the human FVIIa polypeptide. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and well characterized biochemically. Furthermore, the major milk proteins are present in milk at high concentrations (typically from about 1 to 15 g/l). From a commercial point of view, it is clearly preferable to use as the host a species that has a large milk yield. While smaller animals such as mice and rats can be used (and are preferred at the proof of principle stage), within the present invention it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk. See WIPO Publication WO 88/00239 for a comparison of factors influencing the choice of host species. It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489, incorporated herein by reference), beta-lactoglobulin, alpha-lactalbumin, and whey acidic protein. The beta-lactoglobulin (BLG) promoter is preferred. In the case of the ovine beta-lactoglobulin gene, a region of at least the proximal 406 bp of 5′ flanking sequence of the gene will generally be used, although larger portions of the 5′ flanking sequence, up to about 5 kbp, are preferred, such as about 4.25 kbp DNA segment encompassing the 5′ flanking promoter and non-coding portion of the beta-lactoglobulin gene. See Whitelaw et al., Biochem J. 286: 31-39 (1992). Similar fragments of promoter DNA from other species are also suitable.

Other regions of the beta-lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836-840 (1988); Palmiter et al., Proc. Natl. Acad. Sci. USA 88: 478-482 (1991); Whitelaw et al., Transgenic Res. 1: 3-13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta-lactoglobulin gene, is preferred. One such region is a DNA segment which provides for intron splicing and RNA polyadenylation from the 3′ non-coding region of the ovine beta-lactoglobulin gene. When substituted for the natural 3′ non-coding sequences of a gene, this ovine beta-lactoglobulin segment can both enhance and stabilize expression levels of the protein or polypeptide of interest. Within other aspects, the region surrounding the initiation ATG of the sequence encoding the human FVIIa polypeptide is replaced with corresponding sequences from a milk specific protein gene. Such replacement provides a putative tissue-specific initiation environment to enhance expression. It is convenient to replace the entire pre-pro sequence of the human FVIIa polypeptide and 5′ non-coding sequences with those of, for example, the BLG gene, although smaller regions may be replaced.

For expression of a human FVIIa polypeptide in transgenic animals, a DNA segment encoding the human FVIIa polypeptide is operably linked to additional DNA segments required for its expression to produce expression units. Such additional segments include the above-mentioned promoter, as well as sequences which provide for termination of transcription and polyadenylation of mRNA. The expression units will further include a DNA segment encoding a secretory signal sequence operably linked to the segment encoding the human FVIIa polypeptide. The secretory signal sequence may be a native secretory signal sequence of the human FVIIa polypeptide or may be that of another protein, such as a milk protein. See, for example, von Heinje, Nuc. Acids Res. 14: 4683-4690 (1986); and Meade et al., U.S. Pat. No. 4,873,316, which are incorporated herein by reference.

Construction of expression units for use in transgenic animals is conveniently carried out by inserting a sequence encoding the human FVIIa polypeptide into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of the human FVIIa polypeptide, thereby creating a gene fusion that includes the expression control sequences of the milk protein gene. In any event, cloning of the expression units in plasmids or other vectors facilitates the amplification of the human FVIIa polypeptide. Amplification is conveniently carried out in bacterial (e.g. E. coli) host cells, thus the vectors will typically include an origin of replication and a selectable marker functional in bacterial host cells.

The expression unit is then introduced into fertilized eggs (including early-stage embryos) of the chosen host species. Introduction of heterologous DNA can be accomplished by one of several routes, including microinjection (e.g. U.S. Pat. No. 4,873,191), retroviral infection (Jaenisch, Science 240: 1468-1474 (1988)) or site-directed integration using embryonic stem (ES) cells (reviewed by Bradley et al., Bio/Technology 10: 534-539 (1992)). The eggs are then implanted into the oviducts or uteri of pseudopregnant females and allowed to develop. Offspring carrying the introduced DNA in their germ line can pass the DNA on to their progeny in the normal, Mendelian fashion, allowing the development of transgenic herds.

General procedures for producing transgenic animals are known in the art. See, for example, Hogan et al., Manipulating the Mouse Embryo: A Laboratory Manual, Cold Spring Harbor Laboratory, 1986; Simons et al., Bio/Technology 6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49:113-120 (1992); U.S. Pat. Nos. 4,873,191 and 4,873,316; WIPO publications WO 88/00239, WO 90/05188, WO 92/11757; and GB 87/00458, which are incorporated herein by reference. Techniques for introducing foreign DNA sequences into mammals and their germ cells were originally developed in the mouse. See, e.g., Gordon et al., Proc. Natl. Acad. Sci. USA 77: 7380-7384 (1980); Gordon and Ruddle, Science 214: 1244-1246 (1981); Palmiter and Brinster, Cell 41: 343-345 (1985); and Brinster et al., Proc. Natl. Acad. Sci. USA 82: 4438-4442 (1985). These techniques were subsequently adapted for use with larger animals, including livestock species (see e.g., WIPO publications WO 88/00239, WO 90/05188, and WO 92/11757; and Simons et al., Bio/Technology 6: 179-183 (1988). To summarize, in the most efficient route used to date in the generation of transgenic mice or livestock, several hundred linear molecules of the DNA of interest are injected into one of the pro-nuclei of a fertilized egg according to established techniques. Injection of DNA into the cytoplasm of a zygote can also be employed. Production in transgenic plants may also be employed. Expression may be generalized or directed to a particular organ, such as a tuber. See, Hiatt, Nature 344:469-479 (1990); Edelbaum et al., J. Interferon Res. 12:449-453 (1992); Sijmons et al., Bio/Technology 8:217-221 (1990); and European Patent Office Publication EP 255,378.

FVIIa produced according to the present invention may be purified by affinity chromatography on an anti-FVII antibody column. It is preferred that the immunoadsorption column comprise a high-specificity monoclonal antibody. The use of calcium-dependent monoclonal antibodies, as described by Wakabayashi et al., J. Biol. Chem, 261:11097-11108, (1986) and Thim et al., Biochem. 27: 7785-7793, (1988), incorporated by reference herein, is particularly preferred. Additional purification may be achieved by conventional chemical purification means, such as high performance liquid chromatography. Other methods of purification, including barium citrate precipitation, are known in the art, and may be applied to the purification of the FVIIa described herein (see, generally, Scopes, R., Protein Purification, Springer-Verlag, N.Y., 1982). Substantially pure FVIIa of at least about 90 to 95% homogeneity is preferred, and 98 to 99% or more homogeneity most preferred, for pharmaceutical uses. Once purified, partially or to homogeneity as desired, the FVIIa may then be used therapeutically.

Conversion of single-chain FVII to active two-chain FVIIa may be achieved using factor Xlla as described by Hedner and Kisiel (1983, J. Clin. Invest. 71: 1836-1841), or with other proteases having trypsin-like specificity (Kisiel and Fujikawa, Behring Inst. Mitt. 73: 29-42, 1983). Alternatively FVII may be autoactivated by passing it through an ion-exchange chromatography column, such as mono Q.RTM. (Pharmacia Fire Chemicals) or the like (Bjoern et al., 1986, Research Disclosures 269:564-565). The FVIIa molecules of the present invention and pharmaceutical compositions thereof are particularly useful for administration to humans to treat a variety of conditions involving intravascular coagulation.

The compounds of the present invention may have one or more asymmetric centers and it is intended that stereoisomers (optical isomers), as separated, pure or partially purified stereoisomers or racemic mixtures thereof are included in the scope of the invention.

Within the present invention, the TF antagonist may be prepared in the form of pharmaceutically acceptable salts, especially acid-addition salts, including salts of organic acids and mineral acids. Examples of such salts include salts of organic acids such as formic acid, fumaric acid, acetic acid, propionic acid, glycolic acid, lactic acid, pyruvic acid, oxalic acid, succinic acid, malic acid, tartaric acid, citric acid, benzoic acid, salicylic acid and the like. Suitable inorganic acid-addition salts include salts of hydrochloric, hydrobromic, sulphuric and phosphoric acids and the like. Further examples of pharmaceutically acceptable inorganic or organic acid addition salts include the pharmaceutically acceptable salts listed in Journal of Pharmaceutical Science, 66, 2 (1977) which are known to the skilled artisan.

Also intended as pharmaceutically acceptable acid addition salts are the hydrates which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of this invention may form solvates with standard low molecular weight solvents using methods known to the skilled artisan.

The TF antagonist and/or TF inhibitor used in the inventive methods may be administered in pharmaceutically acceptable acid addition salt form or, where appropriate, as a alkali metal or alkaline earth metal or lower alkylammonium salt. Such salt forms are believed to exhibit approximately the same order of activity as the free base forms.

Present treatment of diseases or disorders associated with inflammation, as defined above, including acute and chronic inflammation such as rheumatic diseases (e.g., Lyme disease, juvenile (rheumatoid) arthritis, osteoarthritis, psoriatic arthritis, rheumatoid arthritis and staphylococcal-induced (“septic”) arthritis) includes first line drugs for control of pain and inflammation classified as non-steroidal, anti-inflammatory drugs (NSAIDs). Secondary treatments include corticosteroids, slow acting antirheumatic drugs (SAARDs) or disease modifying (DM) drugs.

In a specific aspect, the present invention is directed to the use of a TF antagonist and/or TF inhibitor in combination with an effective amount of one or more NSAIDs for the treatment of a disease or disorder associated with inflammation, as defined above, including acute and chronic inflammation such as rheumatic diseases (e.g., Lyme disease, juvenile (rheumatoid) arthritis, osteoarthritis, psoriatic arthritis, rheumatoid arthritis and staphylococcal-induced (“septic”) arthritis); and graft versus host disease. NSAIDs owe their anti-inflammatory action, at least in part, to the inhibition of prostaglandin synthesis (Goodman and Gilman in “The Pharmacological Basis of Therapeutics,” MacMillan, 7th Edition (1985)). NSAIDs can be characterized into nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones. Any suitable NSAID and/or combination thereof can be used in such combination methods. The effective dosages of the TF antagonist and/or TF inhibitor can be desirably lessened by the addition of such NSAIDs to the therapeutic regimen and/or combination composition.

In a particular exemplary aspect, the present invention is directed to the use of a TF antagonist and/or TF inhibitor in combination (whether in pretreatment, post-treatment, or concurrent treatment) with any of one or more of the following NSAIDs: epsilon-acetamidocaproic acid, S-adenosylmethionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixim, lefetamine HCl, leflunomide, lofemizole, lotifazole, lysin clonixinate, meseclazone, nabumetone, nictindole, nimesulide, orgotein, orpanoxin, oxaceprolm, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, M861, AD1590, AFP802, AFP860, A177B, AP504, AU8001, BPPC, BW540C, CHINOIN 127, CN100, EB382, EL508, F1044, FK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770. Structurally related NSAIDs having similar analgesic and anti-inflammatory properties to the above NSAIDs are also intended to be encompassed by this group.

In a specific aspect, the inventive methods of the invention can comprise the delivery of a TF antagonist and/or TF inhibitor in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate and sulfasalazine. Structurally related salicylic acid derivatives having similar analgesic and anti-inflammatory properties are also intended to be encompassed by this group.

In another particular aspect, the present invention is directed to the use of TF antagonist and/or TF inhibitor in combination (pretreatment, post-treatment, or concurrent treatment) with any of one or more propionic acid derivatives, prodrug esters or pharmaceutically acceptable salts thereof. The propionic acid derivatives, prodrug esters and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group.

In yet a further aspect, the inventive methods described herein can comprise the delivery of a TF antagonist and/or TF inhibitor in combination (pretreatment, post-treatment or concurrent treatment) with any of one or more acetic acid derivatives, prodrug esters or pharmaceutically acceptable salts thereof. The acetic acid derivatives, prodrug esters and pharmaceutically acceptable salts thereof comprise: acemetacin, alclofenac, amfenac, bufexamac, cinmetacin, clopirac, delmetacin, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, zidometacin and zomepirac. Structurally related acetic acid derivatives having similar analgesic and anti-inflammatory properties are also intended to be encompassed by this group.

In a specific aspect, the present invention is directed to the use of TF antagonist in combination (pretreatment, post-treatment or concurrent treatment) with any of one or more fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefanamic acid, niflumic acid, talniflumate, terofenamate, tolfenamic acid and ufenamate. Structurally related fenamic acid derivatives having similar analgesic and anti-inflammatory properties are also intended to be encompassed by this group.

In additional aspects, the inventive methods described herein can comprise the delivery of an effective amount of a TF antagonist and/or TF inhibitor in combination (pretreatment, post-treatment or concurrent treatment) with an effective amount (which here and in other combination therapy/composition aspects of the invention can be referred to as a “second effective amount”) of any of one or more carboxylic acid derivatives, prodrug esters or pharmaceutically acceptable salts thereof. The carboxylic acid derivatives, prodrug esters and pharmaceutically acceptable salts thereof which can be used comprise: clidanac, diflunisal, flufenisal, inoridine, ketorolac and tinoridine. Structurally related carboxylic acid derivatives having similar analgesic and anti-inflammatory properties are also intended to be encompassed by this group.

In a further aspect, the present invention is directed to the use of TF antagonist and/or TF inhibitor in combination (pretreatment, post-treatment or concurrent treatment) with any of one or more butyric acid derivatives, prodrug esters or pharmaceutically acceptable salts thereof. The butyric acid derivatives, prodrug esters and pharmaceutically acceptable salts thereof comprise: bumadizon, butibufen, fenbufen and xenbucin. Structurally related butyric acid derivatives having similar analgesic and anti-inflammatory properties are also intended to be encompassed by this group.

In yet another aspect, the inventive methods of the invention can include delivery of an effective amount of a TF antagonist and/or TF inhibitor in combination (pretreatment, post-treatment or concurrent treatment) with the delivery of an effective amount any of one or more oxicams, prodrug esters or pharmaceutically acceptable salts thereof. The oxicams, prodrug esters and pharmaceutically acceptable salts thereof comprise: droxicam, enolicam, isoxicam, piroxicam, sudoxicam, tenoxicam and 4-hydroxyl-1,2-benzothiazine 1,1-dioxide 4-(N-phenyl)-carboxamide. Structurally related oxicams having similar analgesic and anti-inflammatory properties are also intended to be encompassed by this group.

In a specific aspect, the present invention is directed to the use of a TF antagonist in combination (pretreatment, post-treatment or concurrent treatment) with any of one or more pyrazoles, prodrug esters or pharmaceutically acceptable salts thereof. The pyrazoles, prodrug esters and pharmaceutically acceptable salts thereof which may be used comprise: difenamizole and epirizole. Structurally related pyrazoles having similar analgesic and anti-inflammatory properties are also intended to be encompassed by this group.

In another exemplary aspect, the inventive methods described herein can comprise the delivery of an effective amount of a TF antagonist and/or TF inhibitor in combination (pretreatment, post-treatment or concurrent treatment) with any of one or more pyrazolones, prodrug esters or pharmaceutically acceptable salts thereof. The pyrazolones, prodrug esters and pharmaceutically acceptable salts thereof which may be used comprise: apazone, azapropazone, benzpiperylon, feprazone, mofebutazone, morazone, oxyphenbutazone, phenylbutazone, pipebuzone, propylphenazone, ramifenazone, suxibuzone and thiazolinobutazone. Structurally related pyrazalones having similar analgesic and anti-inflammatory properties are also intended to be encompassed by this group.

In a further illustrative aspect, in the inventive methods described herein can comprise the delivery of an effective amount of a TF inhibitor and/or TF antagonist in combination (pretreatment, post-treatment or concurrent treatment) with the delivery of an effective amount of any of one or more corticosteroids, prodrug esters or pharmaceutically acceptable salts thereof for the treatment of disease or disorder associated with inflammation, as defined above, including acute and chronic inflammation such as rheumatic diseases (e.g., Lyme disease, juvenile (rheumatoid) arthritis, osteoarthritis, psoriatic arthritis, rheumatoid arthritis and staphylococcal-induced (“septic”) arthritis); and multiple sclerosis. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flunisolide, flucinolone acetonide, fluocinonide, fluorocinolone acetonide, fluocortin butyl, fluocortolone, fluorocortolone hexanoate, diflucortolone valerate, fluorometholone, fluperolone acetate, fluprednidene acetate, fluprednisolone, flurandenolide, formocortal, halcinonide, halometasone, halopredone acetate, hydrocortamate, hydrocortisone, hydrocortisone acetate, hydrocortisone butyrate, hydrocortisone phosphate, hydrocortisone 21-sodium succinate, hydrocortisone tebutate, mazipredone, medrysone, meprednisone, methylprednicolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

In a further specific aspect, the present invention is directed to the use of a TF antagonist and/or TF inhibitor in combination (pretreatment, post-treatment or concurrent treatment) with any of one or more slow-acting antirheumatic drugs (SMRDs) or disease modifying antirheumatic drugs (DMARDS), prodrug esters or pharmaceutically acceptable salts thereof for the treatment of disease or disorder associated with inflammation, as defined above, including acute and chronic inflammation such as rheumatic diseases (e.g., lyme disease, juvenile (rheumatoid) arthritis, osteoarthritis, psoriatic arthritis, rheumatoid arthritis and staphylococcal-induced (“septic”) arthritis); and multiple sclerosis. SAARDs or DMARDS, prodrug esters and pharmaceutically acceptable salts thereof comprise: allocupreide sodium, auranofin, aurothioglucose, aurothioglycanide, azathioprine, brequinar sodium, bucillamine, calcium 3-aurothio-2-propanol-1-sulfonate, chlorambucil, chloroquine, clobuzarit, cuproxoline, cyclophosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine. Structurally related SAARDs or DMARDs having similar analgesic and anti-inflammatory properties are also intended to be encompassed by this group.

In a further exemplary aspect, the present invention provides a method of delivering an effective amount of a TF antagonist and/or TF inhibitor in combination (pretreatment, post-treatment or concurrent treatment) with an effective amount of any of one or more COX2 inhibitors, their prodrug esters or pharmaceutically acceptable salts thereof for the treatment of disease or disorder associated with inflammation, as defined above, including acute and chronic inflammation. Examples of COX2 inhibitors, prodrug esters or pharmaceutically acceptable salts thereof include, for example, celecoxib. Structurally related COX2 inhibitors having similar analgesic and anti-inflammatory properties are also intended to be encompassed by this group.

In a specific aspect, the present invention is directed to the use of a TF antagonist and/or TF inhibitor in combination (pretreatment, post-treatment or concurrent treatment) with any of one or more antimicrobials, prodrug esters or pharmaceutically acceptable salts thereof for the treatment of disease or disorder associated with inflammation, as defined above, including acute and chronic inflammation. Antimicrobials include, for example, ampicillin, amoxycillin, aureomicin, bacitracin, ceftazidime, ceftriaxone, cefotaxime, cephachlor, cephalexin, cephradine, ciprofloxacin, clavulanic acid, cloxacillin, dicloxacillan, erythromycin, flucloxacillan, gentamicin, gramicidin, methicillan, neomycin, oxacillan, penicillin and vancomycin. Structurally related antimicrobials having similar analgesic and anti-inflammatory properties are also intended to be encompassed by this group.

In a specific aspect, the present invention is directed to the use of a TF antagonist in combination (pretreatment, post-treatment or concurrent treatment) with any of one or more of the following compounds for the treatment of disease or disorder associated with inflammation, as defined above, including acute and chronic inflammation: granulocyte colony stimulating factor; thalidomide; BN 50730; tenidap; E 5531; tiapafant PCA 4248; nimesulide; panavir; rolipram; RP 73401; peptide T; MDL 201,449A; (1R,3S)-Cis-1-[9-(2,6-diaminopurinyl)]-3-hydroxy-4-cyclopentene hydrochloride; (1R,3R)trans-1-[9-(2,6-diamino)purine]-3-acetoxycyclopent-ane; (1R,3R)-trans-1-[9-adenyl)-3-azidocyclopentane hydrochloride and (1R,3R)trans-1-[6-hydroxy-purin-9-yl)-3-azidocyclopentane.

In a specific aspect, the present invention is directed to the use of a TF antagonist in combination (pretreatment, post-treatment, or concurrent treatment) with one or more TNF inhibitor for the treatment of disease or disorder associated with inflammation, as defined above, including acute and chronic inflammation. TNF inhibitors include compounds and proteins which block in vivo synthesis or extracellular release of TNF, including the following compounds. In a specific aspect the TNF inhibitor is a TNF alpha inhibitor.

TNF inhibitors include anti-TNF antibodies (e.g., MAK 195F Fab antibody (Holler et al. (1993), 1st International Symposium on Cytokines in Bone Marrow Transplantation, 147; CDP 571 anti-TNF monoclonal antibody (Rankin et al. (1995), British Journal of Rheumatology, 34:334-342, the disclosure of which is hereby incorporated by reference); BAY X 1351 murine anti-tumor necrosis factor monoclonal antibody (Kieft et al. (1995), 7th European Congress of Clinical Microbiology and Infectious Diseases, 9, the disclosure of which is hereby incorporated by reference); CenTNF cA2 anti-TNF monoclonal antibody (Elliott et al. (1994), Lancet, 344:1125-1127 and Elliott et al. (1994), Lancet, 344:1105-1110, the disclosures of which are hereby incorporated by reference).

In a specific aspect, the present invention is directed to the use of a TF antagonist in combination (pretreatment, post-treatment, or concurrent treatment) with the soluble recombinant human Fas antigen or recombinant versions thereof (WO 96/20206 and Mountz et al., J. Immunology, 155:4829-4837; and EP 510 691), the disclosures of which are hereby incorporated by reference. WO 96/20206 discloses secreted human Fas antigen (native and recombinant, including an Ig fusion protein), methods for isolating the genes responsible for coding the soluble recombinant human Fas antigen, methods for cloning the gene in suitable vectors and cell types, and methods for expressing the gene to produce the inhibitors. EP 510 691 teaches DNAs coding for human Fas antigen, including soluble Fas antigen, vectors expressing for said DNAs and transformants transfected with the vector. When administered parenterally, doses of a Fas antigen fusion protein each are generally from 1 micrograms/kg to 100 micrograms/kg.

In another specific aspect, the present invention is directed to the use of a TF antagonist in combination (pretreatment, post-treatment or concurrent treatment) with any of one or more interleukin-1 inhibitors for the treatment of disease or disorder associated with inflammation, as defined above, including acute and chronic inflammation such as rheumatic diseases (e.g., lyme disease, juvenile (rheumatoid) arthritis, osteoarthritis, psoriatic arthritis, rheumatoid arthritis and staphylococcal-induced (“septic”) arthritis); brain injury as a result of trauma, epilepsy, hemorrhage or stroke; and multiple sclerosis. Classes of interleukin-1 inhibitors include interleukin-1 receptor antagonists (any compound capable of specifically preventing activation of cellular receptors to IL-1) such as IL-1 ra, as described below; anti-IL-1 receptor monoclonal antibodies (e.g., EP 623674, the disclosure of which is hereby incorporated by reference); IL-1 binding proteins such as soluble IL-1 receptors (e.g., U.S. Pat. No. 5,492,888, U.S. Pat. No. 5,488,032, U.S. Pat. No. 5,464,937, U.S. Pat. No. 5,319,071 and U.S. Pat. No. 5,180,812, the disclosures of which are hereby incorporated by reference); anti-IL-1 monoclonal antibodies (e.g., WO 9501997, WO 9402627, WO 9006371, U.S. Pat. No. 4,935,343, EP 364778, EP 267611 and EP 220063, the disclosures of which are hereby incorporated by reference); IL-1 receptor accessory proteins, e.g., WO 96/23067 (the disclosure of which is hereby incorporated by reference) and other compounds and proteins which block in vivo synthesis or extracellular release of IL-1.

Interleukin-1 receptor antagonist (IL-1 ra) is a human protein that acts as a natural inhibitor of interleukin-1. Preferred receptor antagonists, as well as methods of making and methods of using thereof, are described in U.S. Pat. No. 5,075,222 (referred to herein as the '222 patent); WO 91/08285; WO 91/17184; AU 9173636; WO 92/16221; WO93/21946; PCT International Application No. US97/02131, which teaches a pharmaceutical composition comprising (a) an effective amount of controlled release polymer (e.g., hyaluronic acid) and (b) an effective amount of an IL-1ra; WO 94/06457; WO 94/21275; FR 2706772; WO 94/21235; DE 4219626, WO 94/20517; and WO 96/22793, the disclosures of which are incorporated herein by reference. The proteins include glycosylated as well as non-glycosylated IL-1 receptor antagonists.

Specifically, three preferred forms of IL-1 ra (IL-1ra.alpha., IL-1ra.beta. and IL-1rax), each being derived from the same DNA coding sequence, are disclosed and described in U.S. Pat. No. 5,075,222 by Hannum et al., entitled “Interleukin-1 Inhibitors.” This U.S. Patent, referred to herein as the '222 patent, is specifically incorporated herein by reference. All three of these interleukin-1 inhibitors possess similar functional and immunological activities. Methods for producing IL-1 inhibitors, particularly IL-1 ras, are also disclosed in the '1222 patent. One disclosed method involves isolating the inhibitors from human monocytes (where they are naturally produced). A second disclosed method involves isolating the gene responsible for coding the IL-1 ras, cloning the gene in suitable vectors and cell types, expressing the gene to produce the IL-1 ras and harvesting the IL-1 ras. The latter method, which is exemplary of recombinant DNA methods in general, is a preferred method of the present invention. In a specific aspect, an IL-1ra contains an N-terminal methionyl group as a consequence of expression in E. coli. The present invention also includes modified IL-1 ras. The modified IL-1 ras include, for example, muteins of such inhibitors in which a cysteine residue is substituted for an amino acid at one or more sites in the amino acid sequence of a naturally-occurring inhibitor. Such muteins may then be ste-selectively reacted with functionalized polyethylene glycol (PEG) units or other sulfhydryl-containing polyethers to create IL-1ra PEG species. PCT Publication No. WO 92/16221 discloses a number of modified IL-1ra species and methods of making such PEG modified inhibitors.

An additional class of interleukin-1 inhibitors includes compounds capable of specifically preventing activation of cellular receptors to IL-1. Such compounds include IL-1 binding proteins, such as soluble receptors and monoclonal antibodies. Such compounds also include monoclonal antibodies to the receptors.

A further class of interleukin-1 inhibitors includes compounds and proteins which block in vivo synthesis and/or extracellular release of IL-1. Such compounds include agents which affect transcription of IL-1 genes or processing of IL-1 preproteins.

The above is by way of example and does not preclude other treatments to be used concurrently with these anti-inflammatory compounds that are known by those skilled in the art or that could be arrived at by those skilled in the art using the guidelines set forth in this specification.

The invention also provides compositions that correspond to the various exemplary combination therapies described herein.

Any of the TF antagonist, TF inhibitor, or combination compositions described herein can be formulated in association with one or more pharmaceutically acceptable carriers (vehicles, diluents, excipients, etc.). The compositions also can include stabilizers, buffers, isotonic agents, preservatives, colorants, flavorants, tablelting agents, wetting agents, solubilizers, solvents, targeting agents, solutes, etc. Such compositions can be referred to as “pharmaceutical compositions.”

Optionally, a pharmaceutical composition of the invention can include a TF antagonist and/or TF inhibitor in combination with one or more other compounds exhibiting anticoagulant activity, e.g., a platelet aggregation inhibitor.

The TF antagonist, TF inhibitor, and/or various combination compositions of the invention may be formulated using any suitable type of pharmaceutically acceptable carrier(s). Such carriers include water, physiological saline, ethanol, polyols, e.g., glycerol or propylene glycol, or vegetable oils. As used herein, “pharmaceutically acceptable carriers” also encompasses any and all solvents, dispersion media, coatings, antifungal agents, preservatives, isotonic agents and the like. Except insofar as any conventional medium is incompatible with the active ingredient and its intended use, its use in the compositions of the present invention is contemplated.

Such compositions may be prepared by conventional techniques and appear in conventional forms, for example, capsules, tablets, solutions or suspensions. The pharmaceutical carrier employed may be a conventional solid or liquid carrier. Examples of solid carriers are lactose, terra alba, sucrose, talc, gelatine, agar, pectin, acacia, magnesium stearate and stearic acid. Examples of liquid carriers are syrup, peanut oil, olive oil and water. Similarly, the carrier or diluent may include any time delay material known to the art, such as glyceryl monostearate or glyceryl distearate, alone or mixed with a wax. The formulations may also include welting agents, emulsifying and suspending agents, preserving agents, sweetening agents or flavoring agents. The formulations of the invention may be formulated so as to provide quick, sustained, or delayed release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or coloring substances and the like, which do not deleteriously react with the active compounds.

The route of administration may be any route, which effectively transports the active compound to the appropriate or desired site of action, such as oral or parenteral, e.g., rectal, transdermal, subcutaneous, intranasal, intramuscular, topical, intravenous, intraurethral, ophthalmic solution or an ointment, the oral route being generally preferred.

If a solid carrier for oral administration is used, the preparation can be tabletted, placed in a hard gelatine capsule in powder or pellet form or it can be in the form of a troche or lozenge. The amount of solid carrier may vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain a compound of formula (I) dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet, which may be prepared by conventional tabletting techniques, contains

| | |
|---|---|
| Core: | |
| Active compound (as free compound or salt thereof) | about 10 mg |
| Colloidal silicon dioxide (Areosil ®) | about 1.5 mg |
| Cellulose, microcryst. (Avicel ®) | about 70 mg |
| Modified cellulose gum (Ac-Di-Sol ®) | about 7.5 mg |
| Magnesium stearate | |
| Coating: | |
| HPMC | approx. 9 mg |
| *Mywacett ® 9-40 T | approx. 0.9 mg |

*Acylated monoglyceride used as plasticizer for film coating.

The compounds of the invention may be administered to a mammal, especially a human in need of such treatment, prevention, elimination, alleviation, and/or amelioration of various thrombolytic or coagulopathic diseases or disorders as mentioned above. Such mammals also include animals, both domestic animals, e.g. household pets, and non-domestic animals such as wildlife.

Usually, dosage forms suitable for oral, nasal, pulmonal, or transdermal administration comprise from about 0.001 mg to about 100 mg, preferably from about 0.01 mg to about 50 mg of the compounds of formula I admixed with a pharmaceutically acceptable carrier or diluent.

The compounds may be administered concurrently, simultaneously, or together with a pharmaceutically acceptable carrier or diluent, whether by oral, rectal, or parenteral (including subcutaneous) route. The compounds are often, and preferably, in the form of an alkali metal or earth alkali metal salt thereof.

Suitable dosage ranges varies as indicated above depending upon the exact mode of administration, form in which administered, the indication towards which the administration is directed, the subject involved and the body weight of the subject involved, and the preference and experience of the physician or veterinarian in charge.

In an additional aspect, the TF antagonist, TF inhibitor, and/or combination composition (e.g., an NSAID, TF antagonist, and TF inhibitor combination composition) of the invention can be used (by delivery of an effective amount thereof) for the reduction of extravascular fibrin deposition in arthritic joints (e.g., in a patient suffering from an arthritic condition). Wherein combination compositions are discussed herein, it should be understood that combination therapies, comprising the separate administration of some or all of the components of any combination composition, also are provided by the invention for similar purposes (even if such combination therapies are not explicitly described).

In another aspect, the TF antagonist, TF inhibitor, and/or combination compositions of the invention can be used (by delivery of an effective amount thereof) for the modulation of synovitis (e.g., the reduction, amelioration, cessation, or prevention of the initiation, development, and/or spread thereof).

In yet another aspect, the invention provides a method of ameliorating the symptoms and/or underlying causes of chronic inflammation comprising delivering to a mammalian host suffering from a chronic inflammatory condition an effective amount of a TF antagonist, TF inhibitor, and/or combination composition of the invention so as to effect the desired physiological change with respect to the chronic inflammatory condition.

In a further aspect, the invention provides a method of reducing the severity and/or spread of progressive joint destruction by applying the various inventive methods described herein. In another aspect, the invention provides a method of treating progressive joint destruction in a mammal by applying such methods.

In a further aspect, the invention provides a method of modulating synoviocyte apoptosis and in angiogenesis by applying the various methods of the invention. In another aspect, the invention provides a method of treating any of the various disorders or conditions described herein which comprises applying any one of the inventive methods in combination with reducing synoviocyte apoptosis and in angiogenesis in the patient human or other mammal by application of any suitable known means therefore.

In an additional aspect, the invention provides a method for reducing TF-initiated fibrin deposition in mammalian tissue comprising applying any of the inventive methods described herein. Typically, such methods are applied to reduce TF-initiated fibrin deposition in extravascular sites. In another aspect, the methods of the invention are applied to reduce deleterious effects associated with oxygen-mediated lung damage, such as may arise in glomerulonephritis and cancer. In another aspect, the invention provides a method of reducing the potentiation of synovial inflammation in a mammal suffering from an arthritic condition.

In another aspect, the invention also provides a method for promoting the sale of a TF antagonist, TF inhibitor, and/or combination composition as described herein (whether being a combination of a TF antagonist and a TF inhibitor, A TF antagonist and an NSAID or other anti-inflammatory agent, or any other combination described herein) comprising distributing information (whether in print (in newspapers, texts, instructions, informational letters, flyers, brochures, peer-reviewed publications, etc.), by internet, by email, by radio, by television, by video, by oral presentation and/or panel presentations, etc.) concerning the use of such compositions in the treatment of any of the disorders described herein (e.g., by key opinion leaders, medical science liaisons, or pharmaceutical sales persons) so as to increase the sale of such compositions for such purposes.

Assays

Inhibition of FVIIa/phospholipids-embedded TF-catalyzed Activation of FX by TF Antagonists FXa Generation Assay (Assay 1):

In the following example all concentrations are final. Lipidated TF (10 pM), FVIIa (100 pM) and TF antagonist or FFR-rFVIIa (0-50 nM) in HBS/BSA (50 mM hepes, pH 7.4, 150 mM NaCl, 5 mM CaCl₂, 1 mg/ml BSA) are incubated 60 min at room temperature before FX (50 nM) is added. The reaction is stopped after another 10 min by addition of ½ volume stopping buffer (50 mM Hepes, pH 7.4, 100 mM NaCl, 20 mM EDTA). The amount of FXa generated is determined by adding substrate S2765 (0.6 mM, Chromogenix, and measuring absorbance at 405 nm continuously for 10 min. IC₅₀ values for TF antagonist inhibition of FVIIa/lipidated TF-mediated activation of FX may be calculated. The IC50 value for FFR-rFVIIa is 51+/−26 pM in this assay.

Inhibition of FVIIa/Cell Surface TF-Catalyzed Activation of FX by TF Antagonists (Assay 2):

In the following example all concentrations are final. Monolayers of human lung fibroblasts WI-38 (ATTC No. CCL-75) or human bladder carcinoma cell line J82 (ATTC No. HTB-1) or human keratinocyte cell line CCD 1102KerTr (ATCC no. CRL-2310) constitutively expressing TF are employed as TF source in FVIIa/TF catalyzed activation of FX. Confluent cell monolayers in a 96-well plate are washed one time in buffer A (10 mM Hepes, pH 7.45, 150 mM NaCl, 4 mM KCl, and 11 mM glucose) and one time in buffer B (buffer A supplemented with with 1 mg/ml BSA and 5 mM Ca²⁺). FVIIa (1 nM), FX (135 nM) and varying concentrations of TF antagonist or FFR-rFVIIa in buffer B are simultaneously added to the cells. FXa formation is allowed for 15 min at 37° C. 50-μl aliquots are removed from each well and added to 50 μl stopping buffer (Buffer A supplemented with 10 mM EDTA and 1 mg/ml BSA). The amount of FXa generated is determined by transferring 50 μl of the above mixture to a microtiter plate well and adding 25 μl Chromozym X (final concentration 0.6 mM) to the wells. The absorbance at 405 nm is measured continuously and the initial rates of colour development are converted to FXa concentrations using a FXa standard curve. The IC50 value for FFR-rFVIIa is 1.5 nM in this assay.

Inhibition of ¹²⁵I-FVIIa Binding to Cell Surface TF by TF Antagonists (Assay 3):

In the following example all concentrations are final. Binding studies are employed using the human bladder carcinoma cell line J82 (ATTC No. HTB-1) or the human keratinocyte cell line (CCD1102KerTr ATCC No CRL-2310) or NHEK P166 (Clonetics No. CC-2507) all constitutively expressing TF. Confluent monolayers in 24-well tissue culture plates are washed once with buffer A (10 mM Hepes, pH 7.45, 150 mM NaCl, 4 mM KCl, and 11 mM glucose) supplemented with 5 mM EDTA and then once with buffer A and once with buffer B (buffer A supplemented with with 1 mg/ml BSA and 5 mM Ca²⁺). The monolayers are preincubated 2 min with 100 μl cold buffer B. Varying concentrations of Mabs (or FFR-FVIIa) and radiolabelled FVIIa (0.5 nM ¹²⁵I-FVIIa) are simultaneously added to the cells (final volume 200 μl). The plates are incubated for 2 hours at 4° C. At the end of the incubation, the unbound material is removed, the cells are washed 4 times with ice-cold buffer B and lysed with 300 μl lysis buffer (200 mM NaOH, 1% SDS and 10 mM EDTA). Radioactivity is measured in a gamma counter (Cobra, Packard Instruments). The binding data are analyzed and curve fitted using GraFit4 (Erithacus Software, Ltd., (U.K.). The IC50 value for FFR-rFVIIa is 4 nM in this assay.

Biosensor Assay (Assay 4):

TF antagonists are tested on the Biacore instrument by passing a standard solution of the TF antagonist over a chip with immobilized TF. This is followed by different concentrations of sTF in 10 mM hepes pH 7.4 containing 150 mM NaCl, 10 mM CaCl₂ and 0.0003% polysorbate 20. Kd's are calculated from the sensorgrams using the integrated Biacore evaluation software.

The present invention is further illustrated by the following examples.

The present invention is not to be limited in scope by the specific aspects disclosed in the examples which are intended as illustrations of a number of aspects of the invention and any aspects which are functionally equivalent are within the scope of this invention. Those skilled in the art will know, or be able to ascertain using no more than routine experimentation, many equivalents to the specific aspects of the invention described herein. These and all other equivalents are intended to be encompassed by the following claims.

### BRIEF DESCRIPTION OF THE DRAWINGS

The present invention is described in further detail in the Example section with reference to the appended drawings, wherein:

FIG. 1. Immunohistochemical analysis of rheumatoid arthritis (RA) synovial tissues. Cryostat tissue sections were stained with specific anti-fibrin (A and G), anti-tissue factor pathway inhibitor (TFPI) (E), anti fibrinogen (F), anti-CD31 (B), and anti TF (C) antibodies. Brown color Indicates positivity. FIG. 1G is a composite image from adjacent microscope fields. Preadsorption of the TF antibody with a 8×molar excess of recombinant TF (specificity control) eliminated the staining.

FIG. 2. Histologic scoring of synovial inflammation and fibrin staining in rheumatoid arthritis (RA) and osteoarthritis (OA) synovial tissues. Specimens were graded separately for inflammation, using a composite score that evaluates synovial lining hyperplasia, lymphocytic infiltration, and sublining layer hyperplasia (0-3 scale for each component; maximum score 9) and fibrin deposition (0-3 scale). The horizontal bars represent the median scores. Differences between groups were analyzed with Fisher's exact test, *=P<0.003.

FIG. 3. Tissue factor (TF) and TF pathway inhibitor (TFPI) measurements in tissue extracts prepared from rheumatoid arthritis (RA) and ostcoarthritis (OA) synovial specimens. TF (a) and TFPI activities (b) were measured using chromogenic assays adjusted for protein concentration of the tissue attract, and were expressed as arbitrary units (AU) per milligram of protein (prot). TF antigenic (Ag) activity was determined by enzyme linked immunosorbent assay (c), and TF mRNA was determined by RNase protection assay and was expressed as AU after normalization with GAPDH mRNA levels (d). Results are expressed as the mean±SEM. Differences between groups were analyzed by Wilcoxon's rank sum test. *=P<0.02 (n) and P<0.05 (b).

FIG. 4. Relationship between tissue factor (TF) and TF pathway inhibitor (TFPI) activity. For TF expression there was a close correlation between the amount of TF mRNA and the corresponding protein. In contrast, no correlation existed between TFactivity and TF antigenic (Ag) levels (b) or between TF activity and TF mRNA levels (c). A strong negative correlation between TF activity and TFPI activity was evident (d). The strength of the relationship between variables was assessed by Spearman's correlation test, a.u.=arbitrary units.

FIG. 5. Associations between tissue factor (TF) activity. TF pathway inhibitor (TFPI) activity, and histologic scores. Among all patients, TF activity was significantly associated with the synovial fibrin score (a) and the inflammation score (b). Conversely, TFPI activity was negatively associated with the fibrin score (c) Associations were deteemined by Fisher's exact test. (a.u.=arbitrary units).

FIG. 6. Clinical and histological features of the knee joints and coagulation times for control mice and mice with antigen induces arthritis (AIA) treated with active site blocked factor VII (FVIIai). The time course of knee joint inflammation in FVIIai treated mice with AIA was measured by external gamma counting of ^{99m}Tc uptake on days 1, 3, and 7 after antigen injection into the right (R) knee (a): Results are expressed as the ratio of ^{99m}Tc uptake in the right (R) arthritic knee joint to that in the left (L) uninflamed knee joint; for each time point the mean and SEM of the ratios are shown. On day 9 of AIA, knee histologic features of control and FVIIai-treated mice were scored for synovial thickness (b), cartilage damage (c), and intraarticular fibrin deposition as evidenced by fibrin immunohistochemistry (d), using an arbitrary scale. Plasma was collected on day 9 of AIA from placebo treated and FVIIai treated mice, and the prothrombin time (PT) in seconds (sec) was determined (e). On day 9 of AIA fibrin deposition, as scored in (d), was associated with the PT in placebo treated and FVIIai treated mice (f). The horizontal bars represent the mean (e) or median (b-d). Statistical significance was tested by Wilcoxon's rank sum test and by fisher's exact test.

FIG. 7. Results of double staining wins anti TF and other antibodies on RA synovium (Staining was performed on synovial tissue specimens obtained from patients with rheumatoid arthritis (RA), using antibodies as described. Anti TF=anti tissue factor).

### EXAMPLES

### Example 1

Human studies: Tissue sampling. Samples of synovial tissue from 12 patients with OA (6 women and 6 men mean±SD age, 74.6±11.7 years) and 10 patients with RA (7 women and 3 men, mean±SD age, 58.6±11.6 years) undergoing joint replacement surgery (knee or hip) were obtained from the Department of Orthopedics (Centre Hospitalier Universitaire Vaudois). OA was diagnosed according to clinical and radiologic criteria, and patients with RA fulfilled at least 4 of the 7 American College of Itheumatology (formerly, the American Rheumatism Association) revised criteria for the classification of RA (Arnett FC. et al. Arhritis Rheum. 31:315-324, 1988). All tissue specimens were cut into small pieces, immediately frozen in pre-cooled hexane, and stored at −70° C. until use. All subsequent analyses were performed on consecutive cryostat sections (1 representative piece per patient).

Histologic scoring. Cryostat sections (5 μm) of OA and RA synovial tissue were analyzed after staining with hematoxylin and eosin (H&E). Only tissue samples with a synovial lining layer were analyzed. All biopsy specimens were scored independently by 2 observers, who were unaware of the diagnosis, by analyzing 4 areas of each section using a 3 point scale. The parameters scored were as follows: a) hyperplasia of the synovial lining layer (grade 1, 1-3 cell layers; grade 2, 4-6 cell layers; and grade 3, >6 cell layers), b) hyperplasia of the synovial sublining layer (3 point scale, 1=mild hyperplasia and 3=severe hyperplasia), and c) lymphocytic infiltration (3 point scale, 1=minimal infiltration and 3=massive infiltration). The mean score for the 4 areas was calculated for each parameter. The overall inflammation score (range 3-9) was determined to be the sum of the scores for the 3 parameters.

Immunohistologic studies. Immunohistologic studies were performed using the following as primary antibody: for TF a rabbit polyclonal anti human TF antibody at 10 μg/ml final concentration (Novo Nordisk, Gentofte, Denmark); for TFPI a mouse monoclonal antibody (mAb) anti human TFPI at 20 μg/ml (American Diagnostica, Greenwich, Conn.); for fibrin an anti human fibrin mAb at 5 μg/ml; for fibrinogen an anti human fibrinogen mAb at 5 μg/ml and for CD31 a mAb at 5 μg/ml (Dako, Zug, Switzerland). Immunohistochemical analysis was performed on air-dried 5 μm cryostat tissue sections, fixed in acetone for 10 minutes at 4° C. before use. Each slide was incubated for 30 minutes with 10% normal human serum, 10% normal goat serum, and 1% bovine serum albumin (BSA). Slides were then overlaid with the primary antibody for 30 minutes at room temperature (for anti-fibrin) or overnight at 4° C. (for the other antibodies). Bound primary antibodies were visualized using the avidin-biotin peroxidase complex (Vectastain Elite ABC kit: Vector, Burlingame Calif.). The color was developed by 3,3′ diaminobenzidine (DAB; Sigma, Buchs, Switzerland) containing 0.01% hydrogen peroxide. After being extensively washed in water, each slide was counterstained with Harris' hematoxylin (Merck, Rahway, N.J.) using Papaniculau's procedure, further dehydrated in graded alcohol, and mounted in Merckoglas (Merck). For specificity control, preadsorption of the antibody was performed by incubating the antibody for 2 hours with an excess of the corresponding antigen. For antigens, we used recombinant human TF for anti-TF antibody, human fibrinogen for anti fibrinogen antibody, and thrombin clotted fibrinogen for anti-fibrin antibody. Because CD31 antigen was not available, we used isotype matched IgG as control. In this latter case, the positive results obtained with the specific antibody was absent. Incubation in which the first antibody was omitted served as a negative control. Fibrin immunostaining in the synovial membrane was graded independently by 2 observers who were unaware of the clinical diagnosis, on a scale of 0 (no staining at all) to 3 (maximum staining).

To characterize TF expressing cells, double staining was performed. The first murine antibody (anti CD3, anti CD31, anti CD68, anti vimentin (Dako, Cambridge, UK) was detected by fluorescein isothiocyanate-labeled anti-mouse antibody; TF staining was revealed by rhodamine-conjugated anti rabbit antibody (Dako).

Preparation of tissue extracts. Cryostat synovial tissue sections containing 200-300 mg of tissue were solubilized overnight at 4° C. under agitation in 1.5 ml of 1% Triton X-100. After centrifugation at 2,000 g for 1 hour at 4° C., supernatants were collected, and the protein concentration was determined using a Bio-Rad Bradford protein assay with BSA as standard. Tissue extracts were then stored at 20° C. until use.

TF and TFPI activity assays. TF activity in tissue extracts from synovial membranes was measured using an Actichrome chromogenic assay (American Diagnostica). Briefly, test samples were incubated with exogenous human FVII, thus allowing formation of the TF/FVII complex. The complex became allosterically activated, and its activity was directly measured by its ability to cleave a highly specific chromogenic substrate for TF/FVIIa complexes. Once cleaved, absorbance at 405 nm of the released p-nitroaniline chromophore was measured, and TF activity was determined from a standard curve of known dilutions of human TF. TF activity for each sample was expressed in arbitrary units (AU) per milligram of protein (0.5 nM of standard TF was arbitrarily defined to have 500 activity units in this assay). TF activity in the absence of added FVII was minimal.

TFPI activity in tissue extracts from synovial membranes was measured using an Actichrome chromogenic assay (American Diagnostica) based on the ability of TFPI to inhibit the catalytic activity of the TF/FVIIa complexes. Briefly, test samples were incubated with TF/FVIIa complexes and FX. The residual activity of the TF/FVIIa complex, reflected by FXa formation, was directly measured using a highly specific chromogenic substrate for FXa. Once cleaved, absorbance at 405 nm of the released p-nitroaniline chromophore was measured, and TFPI activity was determined from a standard curve constructed using known TFPI activity levels. TFPI activity for each sample was expressed in AU per milligram of protein. All assays were performed according to the manufacturer's instructions.

TF enzyme linked immunosorbent assay (CLISA). An Imubind TF sandwich ELISA kit (American Diagnostica), recognizing TF apoprotein, TF, and TF/FVII complexes, was used. Synovial tissue extracts were tested at 2 dilutions (1:5 and 1:20).

TF messenger RNA (mRNA) determination. Cryostat tissue sections (200-300 mg of tissue) from synovial membranes were homogenized in 1 ml of TRIzol reagent (Gibco BRL, Basel, Switzerland), and total RNA extraction were performed according to the manufacturer's instructions. An RNase protection assay was performed using a multiprobe that includes probes for TF and GAPDH. Phosphoimager analysis was performed to quantify mRNA levels of each gene.

Animal studies: Induction of AIA. C57BL/6 mice ages 8-10 weeks (Iffa Credo, L'Arbresle, France) were immunized on day 0 and day 7 by intradermal injection at the base of the tail, with 100 μg methylated BSA (mBSA) (Sigma) emulsified in 0,1 ml Freund's complete adjuvant containing 200 μg mycobacterial strain H37RA (Difco, Basel, Switzerland). On the same days, heat killed Bordetella pertussis at 2×10⁹ organisms (Berna Biotech, Berne, Switzerland) was injected intraperitonically as an additional adjuvant. Arthritis was induced on day 21, by intraarticular injection of 100 μg mBSA in 10 μl sterile phosphate buffered saline (PBS) into the right knee; the left knee was injected with sterile PBS alone. Institutional approval for these experiments was obtained.

Isotopic quantification of joint inflammation. Joint inflammation was measured by isotopic uptake in the knee joint, as previously described (Kruijsen M W. et al. Agents Actions 11:640-42, 1981). Briefly, mice were first anesthetized using methoxyflurane and then injected subcutaneously in the neck region with 10 μCi ^{99m}Tc. Accumulation of the isotope in the knee was determined after 15 minutes by external gamma counting. The ratio of ^{99m}Tc uptake in the inflamed arthritic knee versus ^{99m}Tc uptake in the contralateral (control) knee was calculated. A ratio higher than 1.1 indicated joint inflammation.

Histologic grading of arthritis. At least 6 mice per group were killed, and the knee joints were dissected and fixed in 10% buffered formalin for 7 days. Fixed tissues were decalcified in 15% EDTA for 3 weeks, dehydrated, and embedded in paraffin. Sagittal sections (8 μm) of the whole knee joint were stained with Safranin O and counterstained with fast green/iron hematoxylin. Histologic sections were graded independently by 2 observers, using a scale of 0-3 (0 normal thickness of the lining layer and sublining tissue (<40 μm), 1 ≈200 μm, 2≈600 μm, and 3≈maximum thickness, (≈1,500 μm)). The degree of thickness reflects underlying inflammatory cell infiltration and hyperplasia. Cartilage proteoglycan depletion, as measured by Safranin O staining intensity, was scored on a scale of 0 (fully stained cartilage) to 3 (totally unstained cartilage). For each histopathologic parameter, at least 8 arthritic knee joints (and at least 3 sections per joint) were examined. The mean of the scores obtained by the 2 observers was calculated.

Fibrin immunohistochemistry: Fibrin immunostaining was performed on knee joint paraffin sections, as described previously (8). Fibrin staining in the synovial membrane was graded independently by 2 observers, who were unaware of the treatment used, on a scale of 0 (no fibrin at all) to 6 (maximum fibrin staining).

Systemic treatment with active site blocked FVIIa (FVIIai). FVIIai was prepared from purified recombinant FVIIa that had been incubated with D-Phe-L-Phe-L-Arg chloromethyl ketone (Sorensen BB. et al. J. Biol. Chem. 272:11863-8,1997). FVIIai was delivered in mice by mini osmotic pumps. Briefly, immunized mice were anesthetized, their backs were shaved, and miniosmotic pumps (model 2002; Alza, Palo Alto, Calif.) filled with a buffered solution of 2.2 mg/ml FVIIai were implanted subcutaneously into their backs (one mini pump per animal). The insertion sites were then closed by sutures. The pumps deliver 0.5 μl/hour, so the mice received 26 μg/day. In control animals, buffer-filled mini pumps were implanted. On day 3 of FVIIai infusion, arthritis was induced by intraarticular injection of mBSA. After 9 days of arthritis, the mice were killed.

Prothrombin time (PT) measurements. Blood was collected from the tail vein or from the interior vena cave of anesthetized animals in 0.12 M trisodium citrate (1 volume of citrate to 9 volumes of blood). Blood samples were centrifuged at 1,500 g for 15 minutes at 4° C., and plasma samples were stored at 20° C. until use. For determination of the PT, 50 μl of 5 fold diluted plasma in Owren's buffer (sodium diethylbarbiturate buffer, pH 7.35) was used. After addition of 100 μl of a thromboplastin reagent (RecombiPlastin Ortho; Almedica, Galmiz, Switzerland), time to thrombus formation was recorded using a microcoagulometer (DiaLine, Itingen, Switzerland).

Plasma FVIIai antigenic concentration. The concentration of FVIIai in citrated plasma was measured by a commercially available ELISA kit designed for human FVII (Asserachrom VII:Ag: Diagnostics Stago. Asnieres-sur-Seine, France), which crossreacts with, human FVIIai but not with murine FVII/FVIIa. The plasma concentration of FVIIai was calculated according to a FVIIai standard curve.

Statistical analysis. Data are reported as the median or the mean±SEM values, as indicated. Differences between the groups were analyzed with the nonparametric Wilcoxon's rank sum test (for continuous variables) or with Fisher's exact test (for categoric variables). Associations were determined with Spearman's rank correlation test or with Fisher's exact test, as appropriate. The Bonferrroni adjustment was applied according to the number of independent parameters tested. For all analyses, P values less than 0.05 were considered significant.

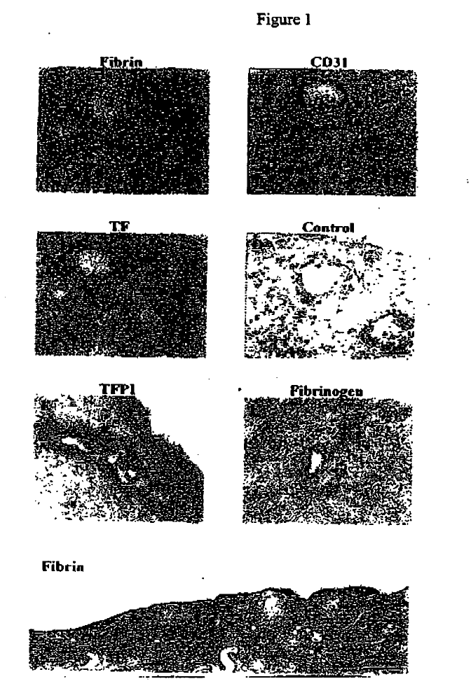

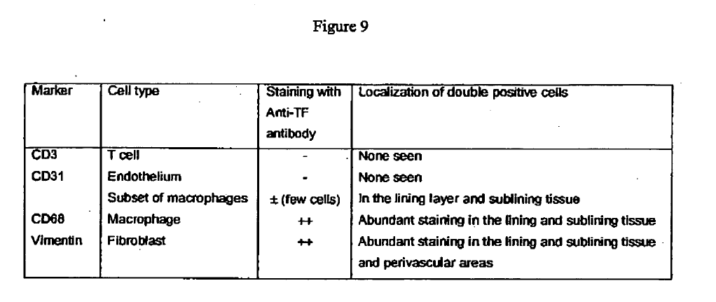

Expression of TF, TFPI, and fibrin in RA Synovial membranes. Antibodies specific for TF, TFPI, and fibrin were used to stain sections of RA synovial membranes. FIG. 1 shows a representative example of the distribution of the different antigens. TF staining was mainly perivascular and patchily distributed in interstitial layers. TF expression was demonstrated in fibroblasts, smooth muscle cells, and macrophages, but not in endothelial cells (FIG. 1C). The latter observation was confirmed by double staining with cell specific markers; the results are summarized in FIG. 7. TF immunohistochemistry was specific, because preadsorption of the TF antibody with an 8×molar excess of recombinant TF eliminated most of the staining (see FIG. 1D). TFPI staining was observed on endothelial and subendothelial cells around blood vessels but was not found in all vessels. Fibrin was prominent in the synovial lining layer and in the deeper layers of the synovium. Fibrin was mainly associated with extracellular matrix but not with vascular or perivascular areas (FIGS. 1A and G). Fibrinogen staining was faint and evenly distributed throughout the synovial tissue, in contrast to the clearly localized staining seen with other antibodies (FIG. 1F).

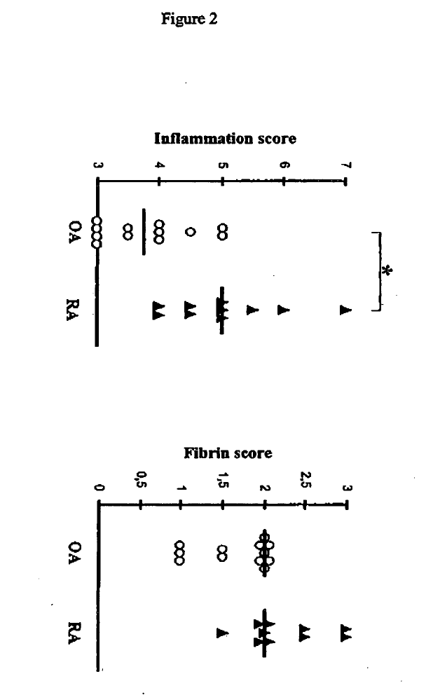

Histologic scoring of inflammation and fibrin deposition in RA and OA synovia. Consecutive sections from 10 patients with RA and 12 patients with OA were stained with H&E anti with an anti fibrin antibody and were scored for the degree of inflammation and the intensity of fibrin staining, respectively. As expected, there was significantly more inflammation in RA synovial membranes (P<0.003), and this was paralleled by more intense fibrin staining. FIG. 2 illustrates the differences between OA and RA synovial membranes.

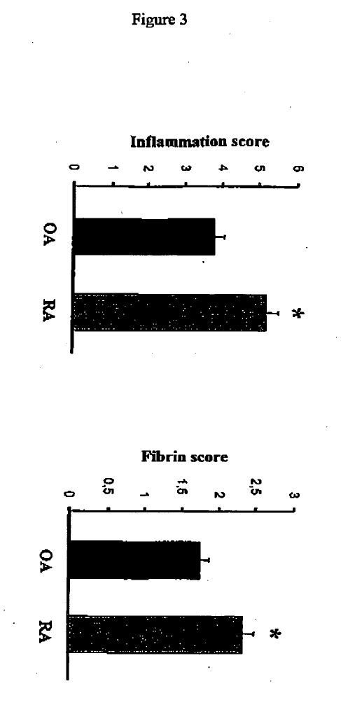

Functional activities of TF and TFPI in RA and OA synovia. Synovial membrane extracts, prepared from the samples on which the histologic analyses were performed, were assessed for the functional activities of TF and TFPI. TF activity was detected in 7 (70%) of 10 RA synovial tissues and in only 3 (25%) of 12 OA samples. Moreover, TF activity was higher in the RA group (range 11.9-138.7 AU/mg protein) compared with the OA group (6.4-76.2 AU/mg protein), with mean TF activity significantly increased in RA synovia (42.9 versus 7.6 AU/mg protein; P<0.02) (FIG. 3a). In contrast, mean TFPI activity in RA tissue was diminished compared with that in OA tissue (2.35 versus 3.57 AU/mg protein; P<0.05) (FIG. 3b).

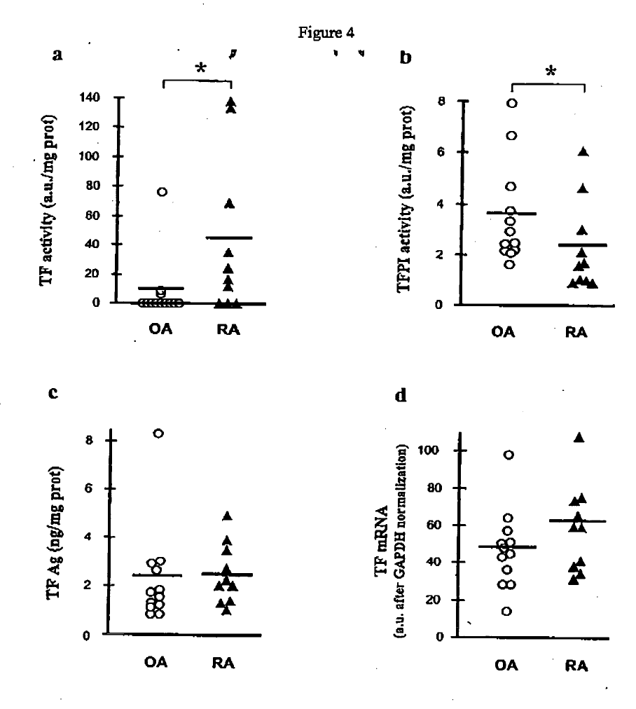

Relationship between functional, antigenic, and mRNA levels of TF. The antigenic and mRNA levels of TF (FIGS. 3c and d) in the RA and OA synovial tissues were determined. Antigenic and mRNA levels of TF were similar in both groups (FIGS. 3c and d). A positive correlation between mRNA and antigenic levels of TF, as measured by ELISA, was observed (FIG. 4a) (r=0.5, P=0.018). In contrast, TF functional activity did not correlate with antigenic (FIG. 4b) and mRNA concentrations of TF (FIG. 4c). The discrepancy between functional activity and antigenic concentration may be explained by TFPI. TF activity showed a significant negative correlation with local TFPI activity (FIG. 4d) (rₛ=−0.87, P<0.0001; subgroup analysis for RA, rₛ=−0.9019, P=0.0004; for OA, rₛ=−0.707, P=0.012).

To investigate whether TFPI activity is the major determinant of TF activity, partial correlation analyses were performed between TF, TFPI activity and TF mRNA levels. The correlation between TF and TFPI was −0.518, and the partial correlation was −0.485. The correlation between TF mRNA and TF activity was weak at 0.181, and the partial correlation was 0.041. These findings suggest that synovial procoagulant activity, the product of the balance between local concentrations of functional TF and TFPI, is determined mainly by TFPI levels.

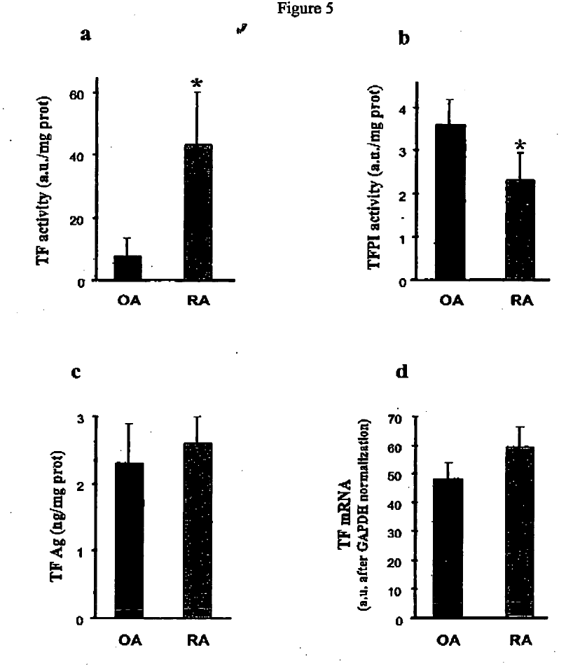

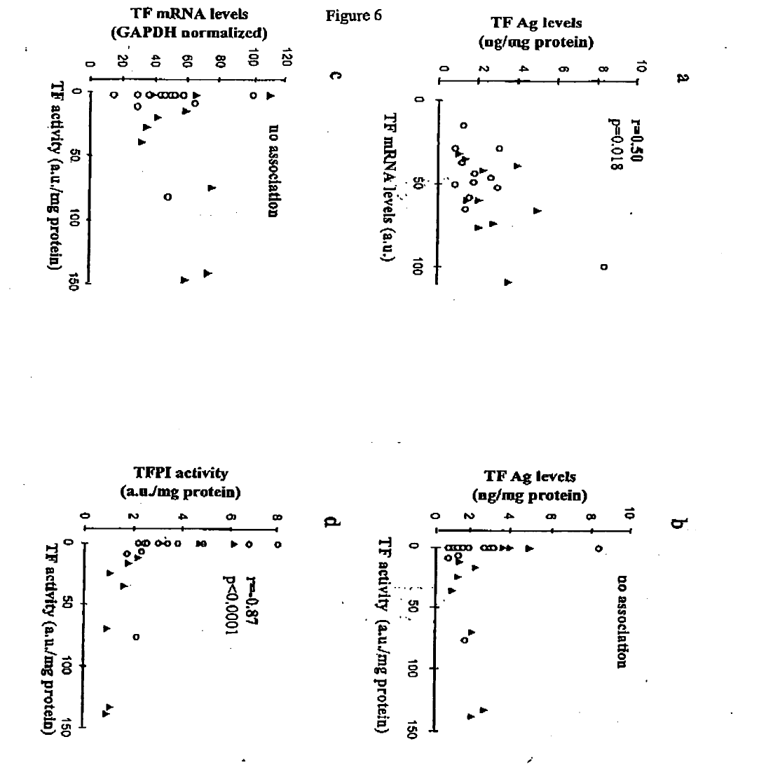

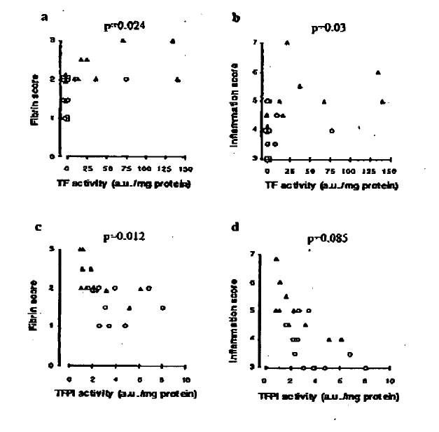

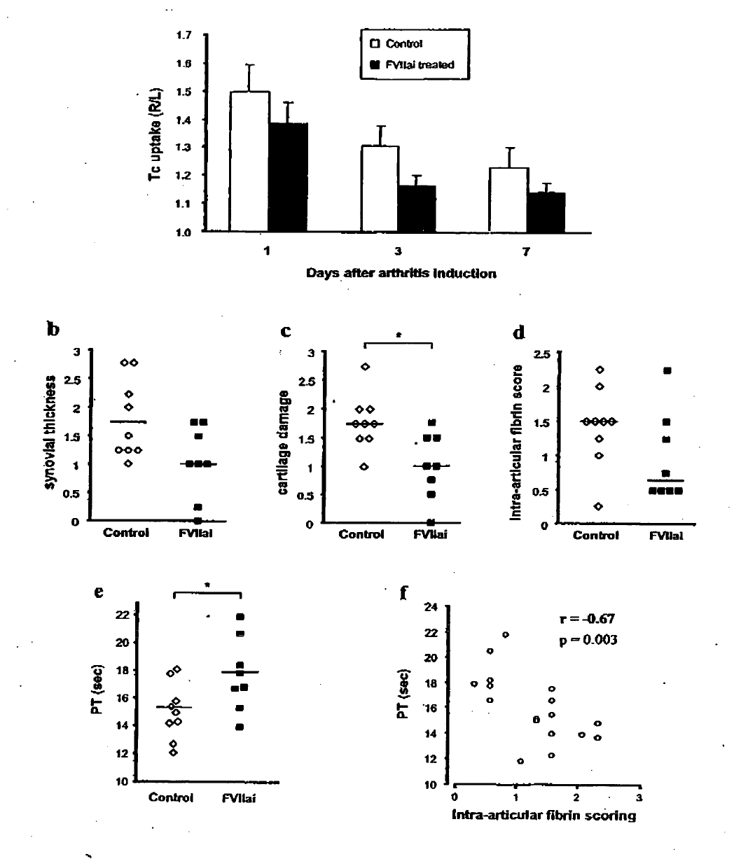

Associations between functional activities of TF and TFPI and histologic scoring. Associations were TFPI sought between histologic scoring of inflammation and fibrin deposition, and the functional levels of TF and TFPI measured in the synovial membranes. In all patients, TF activity was significantly associated with the fibrin score (P=0.024) (FIG. 5a) and with the inflammation score (P=0.03) (FIG. 5b). Conversely, TFPI activity was negatively associated wit fibrin scores (P=0.012) (FIG. 5c). Amelioration of AIA in mice by inhibition of TF pathway. We proceeded to investigate whether blockade of the TF pathway by FVIIai may reduce articular inflammation in the AIA model of inflammatory arthritis. FVIIai was administered by osmotic mini-pumps implanted in the mice. Treatment started 3 days before intraarticular injection of mBSA and continued for 9 days. Vehicle alone was administered to the control group. Four days after mini-pump implantation, there was a sustained level of plasma FVIIai, as detected by ELISA, in 9 treated mice (99.3±7.3 ng/ml), whereas FVIIai was undetectable in 10 control mice (0.2±0.1 ng/ml). Administration of FVIIai led to decreased uptake of ^{99m}Tc on days 1, 3, and 7 (FIG. 6a) and to attenuation of histopathologic features of AIA on day 9 when compared with controls (FIGS. 6b and c), although only the decrease in cartilage damage reached significance (p<0.04, controls (n=9) versus treated mice (n=8). The intraarticular fibrin deposition score was also reduced by 40% in the treated mice, although this reduction did not reach significance (FIG. 6d). The presence of plasma FVIIai was paralleled by a significantly prolonged PT when compared with control animals (FIG. 6e). Finally, there was a strong negative association between the PT and intraarticular fibrin deposition (FIG. 6f).

Several aspects of this invention originated from the inventors' discovery of the contributions of tissue factor (TF) and of its inhibitor, TF pathway inhibitor (TFPI), in arthritis, as illustrated by the detailed experiments described in the foregoing Example section. Synovial tissue specimens obtained from 10 patients with rheumatoid arthritis (RA) and 12 patients with osteoarthritis (OA) were scored histologically for inflammation and fibrin content. TF and TFPI levels were assayed at antigenic and functional levels. TF messenger RNA (mRNA) levels were determined using RNase protection assays. The effect of TF inhibition in murine antigen-induced arthritis (AIA) was assessed by administering systemically active site blocked factor VIIa (FVIIai). Functional TF activity was significantly increased in synovial membranes from RA patients compared with those from OA patients. In contrast, no difference in TF mRNA and TF antigenic levels was observed between these 2 groups: This discrepancy may be accounted for by TFPI, because we observed a negative correlation between TF activity and TFPI activity. There was a significant difference between the RA and OA groups in terms of synovial inflammation, with more inflammation observed in the RA group. Most importantly, TF activity was dissociated with fibrin scoring (P=0.024) and with histologic inflammation scoring (P=0,03). In AIA, inhibition of TF induced coagulation by FVIIai led, on day 9 of arthritis, to decreased synovial thickness and decreased articular cartilage damage, although only the latter difference between controls and treated mice in these experiments reached significance (P<0.04). Finally, in FVIIai treated mice, there was a strong negative association between the prothrombin time and intraarticular fibrin deposition.

The foregoing Example section demonstrates the examination of the expression of TF and TFPI in the synovia of patients with RA, in which chronic synovitis is prominent, and in those with osteoarthritis (OA), which is characterized by limited synovial inflammation. Furthermore, these Examples demonstrate the examination of whether inhibition of the TF pathway of coagulation may influence the severity of antigen induced arthritis (AIA), an experimental model of murine arthritis that replicates some of the features of RA. The findings from these Examples confirm that activation of the coagulation cascade is intimately associated with synovial inflammation and suggest that interventions directed against coagulation mechanisms are beneficial in the treatment of various forms of arthritis.

Immunostaining of RA tissues showed TF staining around small blood vessels and on macrophage-like cells. This demonstrates that there is local synthesis of TF in cell types that are found within the joint. RNase protection assays to quantify local transcription activity further confirmed this. The correlation between the TF mRNA levels and the results obtained by ELISA suggests that local synthesis within the synovial membrane accounts for the bulk of TF in the joint. Although antigenic and mRNA levels of TF within the RA joint were not very different from those in the OA joint, significantly elevated levels of TF activity in RA compared with OA were observed. This dichotomy could be accounted for by the local balance between TF and its inhibitor TFPI. We observed that functional TF levels were negatively correlated with the local activity of TFPI such that the overall balance between the procoagulant molecule and its inhibitor resulted in a higher level of TF procoagulant activity in the RA joint. Partial correlation analysis showed that TF activity is mainly determined by TFPI activity and not by the level of TF mRNA. The inventors have found that in RA, TF activity is increased and is linked to fibrin deposition. Data obtained by the inventors further demonstrated that blocking the proximal part of the coagulation cascade by an inhibitor of TF influences joint inflammation. The inventors of the present invention were able to show functional interference with the coagulation pathway by administering FVIIai, and this reduced joint inflammation and cartilage damage scores. Moreover, in AIA, ^{99m}Tc uptake showed a trend toward reduced inflammation in the treated group. Thus, the therapeutic benefit of TF inhibitors can be extended to an arthritis model. The demonstration of prolongation of the PT in treated animals showed that treatment had a biologic effect.

In conclusion, the data presented by the inventors herein shows that inhibition of TF activity may be used in the treatment of inflammatory arthritides, such as RA. Moreover, the results of the present invention show that TF expression in arthritic synovial tissue favors extravascular coagulation and plays a role in inflammation in RA. The results of the present invention further show that TF inhibitors and TF antagonists can have a therapeutic value in the treatment of inflammatory conditions.

All references, including publications, patent applications, and patents, cited herein are hereby incorporated by reference to the same extent as if each reference were individually and specifically indicated to be incorporated by reference and were set forth in its entirety herein (to the maximum extent permitted by law).

Any combination of the above-described elements in all possible variations thereof is encompassed by the invention unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising,” “having,” “including,” “containing”, and the like are to be construed as open-ended terms (i.e., meaning “including, but not limited to,”) unless otherwise noted and should be read as encompassing the phrases “consisting”, “substantially comprised of,” and “consisting essentially of” (e.g., where a disclosure of a composition “comprising” a particular ingredient is made, it should be understood that the invention also provides an otherwise identical composition characterized by, in relevant part, consisting essentially of the ingredient and (independently) a composition consisting solely of the ingredient).

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

Preferred aspects of this invention are described herein. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

What is claimed is:

1. A method for reducing inflammation in a tissue of a mammal affected by a rheumatic condition comprising delivering an effective amount of a TF antagonist to the mammal so as to reduce inflammation in the tissue.

2. The method of claim 1, wherein the mammal is a human suffering from an inflammation-related condition and the method is used as a therapeutic regimen against the disease.

3. The method of claim 2, wherein the TF antagonist is an inactive FVIIa polypeptide.

4. The method of claim 3, wherein the inactive FVIIa polypeptide is native human FVIIa or a fragment thereof catalytically inactivated in the active site of the FVIIa polypeptide.

5. The method of claim 4, wherein the inactive FVIIa polypeptide is native human FVIIa catalytically inactivated in the active site of the FVIIa polypeptide.

6. The method of claim 2, wherein the TF antagonist comprises an antibody against TF.

7. The method of claim 6, wherein the antibody comprises a human monoclonal antibody against human TF.

8. The method of claim 2, wherein the TF antagonist comprises more than one binding site for TF.

9. The method of claim 2, wherein the method further comprises administering an effective amount of a TNF inhibitor, a second TF antagonist, or a combination thereof.

10. The method of claim 2, wherein the method is used as part of a therapeutic regimen against osteoarthritis in the human.

11. The method of claim 2, wherein the method is used as part of a therapeutic regimen against rheumatoid arthritis in the human.

12. The method of claim 2, wherein the method is used as part of a therapeutic regiment against ankylosing spondylitis, bursitis, fibromyalgia, gout, infectious arthritis, psoriatic arthritis, reactive arthritis, Reiter's Syndrome, scleroderma, systemic lupus erythematosus, tendinitis, Lyme disease, carpal tunnel syndrome, Raynaud's Phenomenon, or a combination of any thereof.


SEQUENCE LISTING (1 sequence)

SEQ ID NO: 1 - protein, 406 aa, Human
  MISC_FEATURE (1)..(406): Xaa=carboxyglutamic acid (gamma-carboxyglutamate)
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP